(19) United States
(12) Patent Application Publication
(10) Pub. No.: US 2026/0232723 A1
(43) Pub. Date: Aug. 13, 2026

(54) NITRIC OXIDE-GENERATING COMPOSITIONS, KITS AND COMBINATIONS FOR USE IN THE TREATMENT, AMELIORATION OR PREVENTION OF RESPIRATORY DISEASES OR DISORDERS

(71) Applicant: THIRTY RESPIRATORY LIMITED, London (GB)

(72) Inventors: Hugh Semple MUNRO, London (GB); Keith LIPMAN, London (GB); Nicholas David BOOTE, London (GB)

(21) Appl. No.: 19/156,193

(22) PCT Filed: Feb. 14, 2024
(86) PCT No.: PCT/GB2024/050390

§ 371 Date: Aug. 13, 2025

(30) Foreign Application Priority Data
Feb. 14, 2023 (GB) 2302117.3

(51) Int. Cl. A61K 33/00; A61K 31/194; A61K 47/26

(52) U.S. Cl. CPC A61K 33/00; A61K 31/194; A61K 47/26

(57) ABSTRACT

The invention relates to nitric oxide-generating compositions, kits and combinations for the treatment, amelioration or prevention of respiratory diseases or disorders, and methods of treating, ameliorating or preventing such diseases or disorders with the compositions, kits and combinations.

The present invention relates to nitric oxide-generating compositions, kits and combinations for the treatment, amelioration or prevention of respiratory diseases or disorders, and methods of treating, ameliorating or preventing such diseases or disorders with the compositions, kits and combinations.

### BACKGROUND

Nitric oxide (NO) and nitric oxide precursors have been extensively studied as potential pharmaceutical agents. There remain substantial problems in connection with the efficient generation and delivery of nitric oxide, other oxides of nitrogen and precursors thereof to organisms and cells for treatment. A widely adopted system for the generation of nitric oxide relies on the acidification of nitrite salts using an acid to produce initially nitrous acid (HNO₂), which nitrous acid then readily decomposes to nitric oxide and nitrate ions with hydrogen ions and water. The decomposition can be represented by the following balanced equation (1):

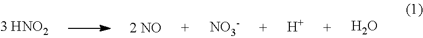

The acid and nitrite salt are typically provided as separate aqueous solutions at pre-determined concentrations and combined at the point of need to prevent the release of nitric oxide before required.

The generation of nitric oxide from the acidification of a nitrite salt depends, at least in part, on the pH of the combined mixture. At a very general level, the more acidic (lower pH) the acid solution that is combined with nitrite salt, the greater the generation of nitric oxide from the combination. As the pH of the acid solution increases, the amount of nitric oxide generated generally decreases.

The use of very low pH (strongly acidic) acid solutions however can create tolerance issues when the generation of nitric oxide is required in a physiological environment, e.g. in the treatment of a disease or disorder. For example, very acidic solutions can potentially cause damage to internal tissues (e.g. lungs for treatment of lower respiratory disorders).

There is therefore a need to provide nitric oxide-generating compositions by the acidification of nitrite salts which efficiently generate nitric oxide in a physiological environment.

### SUMMARY OF THE INVENTION

The present inventors have surprisingly found nitric oxide-generating compositions that are more effective when used to generate nitric oxide in physiological conditions. The nitric oxide-generating compositions of the present invention include a buffer where the buffer capacity of the buffer is higher than conventional buffered acids in nitric oxide-generating compositions.

In general, the acidity of nitric oxide-generating compositions decreases after the initiation of the acidification of the nitrite salt. As such, the effective output from the nitric oxide-generating composition decreases over time. Without wishing to be bound by theory, the present inventors believe that using a buffer with such a buffer capacity allows for the nitric oxide-generating compositions to acidify the nitrite sufficiently through the acid of the buffer, while also allowing the nitric oxide-generating composition to remain at a suitable pH for generating nitric oxide for longer than conventional compositions, especially in physiological conditions.

In general, a buffer is a solution that can resist pH change upon the addition of an acidic or basic components. It is able to neutralize small amounts of added acid or base, thus maintaining the pH of the solution at a relatively stable level. Buffer solutions also typically maintain pH levels on moderate dilution. Buffer solutions have a working pH range and capacity which dictate how much acid/base can be neutralized before pH changes, and the amount by which it will change.

By increasing the buffer capacity of the buffer, the nitric oxide-generating compositions of the present invention may resist increases in pH from both the progression of the acidification of nitrite and from factors from the physiological environment (which typically involve physiological or biological buffers, such as phosphate buffers and/or may dilute the composition), while maintaining generation of nitric oxide through the acidification of the nitrite salt.

In a first aspect, the present invention provides a nitric oxide-generating composition for the treatment, amelioration or prevention of respiratory diseases or disorders, the composition comprising:
a) One or more nitrite salts; and
b) A buffer system comprising at least one acid and at least one conjugate base and water, wherein the buffer system has a pH in the range of 4.6 to 6.0, and wherein the buffer capacity β of the buffer system is at least 0.06 as calculated by the equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of buffer system;
C_{buf} is the buffer concentration in the composition; and
Kₐ is the dissociation constant of the acid at 25° C.

The acid and its conjugate base used as the buffer may be monoprotic or polyprotic.

When the acid is monoprotic, the component

$$\frac{{C}_{buf} {K}_{a} [ {H}^{+} ]}{{( {K}_{a} + [ {H}^{+} ] )}^{2}} )$$

of the equation is calculated based on a single acid dissociation constant. When the acid is polyprotic, the component

$$\frac{{C}_{buf} {K}_{a} [ {H}^{+} ]}{{( {K}_{a} + [ {H}^{+} ] )}^{2}} )$$

is calculated for each acid dissociation constant and summed (as indicated by the Z symbol). For example, citric acid has three dissociation constants and so this component is calculated for Kₐ₁, Kₐ₂ and Kₐ₃ and the resulting values added together as the value for the component

$$\sum \frac{{C}_{buf} {K}_{a} [ {H}^{+} ]}{{( {K}_{a} + [ {H}^{+} ] )}^{2}} ) .$$

Similarly, when the buffer includes a single acid and conjugate base pair as the buffer, the component

$$\sum \frac{{C}_{buf} {K}_{a} [ {H}^{+} ]}{{( {K}_{a} + [ {H}^{+} ] )}^{2}} )$$

is calculated based on the single acid and conjugate base pair. Where the buffer includes more than one acid and conjugate base pair as the buffer, the component

$$\sum \frac{{C}_{buf} {K}_{a} [ {H}^{+} ]}{{( {K}_{a} + [ {H}^{+} ] )}^{2}} )$$

is calculated based on each acid and conjugate base pair (taking into account of polyprotic acids, where applicable) and summed.

The buffering to a pH of 4.6 to 6.0 and with a buffer capacity β of at least 0.06 as defined in equation (2) may provide a more effective nitric oxide-generating composition in physiological conditions.

The present inventors have also found that the useful range of buffer capacities can be varied depending on the pH of the buffer system to account for a decreasing efficacy of compositions at or above a pH of 6. A lower buffer capacity may be used when the pH of the buffer is significantly lower than a pH of 6. Meanwhile, a higher buffer capacity may be used when the pH of the buffer is closer to a pH of 6. The present inventors have therefore termed a measure for buffer capacity in the context of the present invention, the relevant useful buffer capacity (RUβ), where the RUβ is the integral of β with respect to pH between the pH of the buffer system (pH_i) and a pH of 6.00 and β is the buffer capacity of the buffer system as calculated by the equation (2). The RUβ may be expressed as equation (3).

$$R U β = {\int }_{pH \_ i}^{p H = 6.00} β ( pH ) d ( pH ) \qquad ( 3 )$$

In a second aspect, the present invention provides a nitric oxide-generating composition for the treatment, amelioration or prevention of respiratory diseases or disorders, the composition comprising:
a) One or more nitrite salts; and
b) A buffer system comprising at least one acid, at least one conjugate base and water, wherein the buffer system has a pH in the range of 4.6 to 6.0, and wherein the relevant useful buffer capacity RUβ of the buffer system is at least 0.04, where the Ruβ is defined as in equation (3)

$$R U β = {\int }_{pH \_ i}^{p H = 6.00} β ( pH ) d ( pH ) \qquad ( 3 )$$

As the integral of β with respect to pH between the pH of the buffer system (pH_i) and a pH of 6.00 using the trapezoidal method at pH intervals of 0.01 and β is the buffer capacity of the buffer system as calculated by the equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of the buffer system;
C_{buf} is the buffer concentration in the composition; and
Kₐ is the dissociation constant of the acid at 25° C.

In a third aspect, the present invention provides a kit for providing a nitric oxide-generating composition for the treatment, amelioration or prevention of respiratory diseases or disorders, the kit including:
a) A nitrite salt component including one or more nitrite salts;
b) An acid component including at least one acid; and

Wherein the kit further includes at least one conjugate base to form a buffer system with the acid component such that the buffer system has a pH of 4.6 to 6.0, and wherein the buffer capacity β of the buffer system is at least 0.06 as calculated by the equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of the buffer system;
C_{buf} is the buffer concentration in the nitric oxide-generating composition; and
Kₐ is the dissociation constant of the acid at 25° C.

In a fourth aspect, the present invention provides a kit for providing a nitric oxide-generating composition for the treatment, amelioration or prevention of respiratory diseases or disorders, the kit including:
a) A nitrite salt component including one or more nitrite salts; and
b) An acid component including at least one acid; and
   Wherein the kit further includes at least one conjugate base to form a buffer system with the acid component such that the buffer system has a pH of 4.6 to 6.0, and the relevant useful buffer capacity RUβ of the buffer system is at least 0.04, where the Ruβ is defined as in equation (3):

$$RU β = {\int }_{pH \_ i}^{pH = 6.} β ( pH ) d ( pH ) \qquad ( 3 )$$

As the integral of β with respect to pH between the pH of the buffer system (pH_i) and a pH of 6.00 using the trapezoidal method at pH intervals of 0.01 and β is the buffer capacity of the buffer system as calculated by the equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of the buffer system;
C_{buf} is the buffer concentration in the nitric oxide-generating composition; and
Kₐ is the dissociation constant of the acid at 25° C.

In the kit of the third and fourth aspects, the conjugate base or bases may be included in the nitrite component, the acid component or a conjugate base component. In particular embodiments, the conjugate base or bases are included in the acid component such that the acid component includes a buffer system including at least one acid and at least one conjugate base. Water required to form the buffer system may be present in one or more of the nitrite component, the acid component or a conjugate base component (if present). When water is present in either the nitrite component or the acid component or water is present in both acid and nitrite components, the nitrite and acid components are separate components in the kit. The nitrite and acid components can then be combined at the point of use to initiate the acidification of nitrite. Alternatively, the kit of the third and fourth aspects may include instructions with the volume of aqueous media (e.g. water) to be added to form the buffer system. When the acid and nitrite components do not include water (namely are solid components), the acid and nitrite components may be a mixture of the acid and nitrite components in the kit. Aqueous media (e.g. water) may then be added to the combined acid and nitrite components at point of use to initiate the acidification of nitrite.

In a fifth aspect, the present invention provides a nitric oxide-generating composition for the treatment, amelioration or prevention of respiratory diseases or disorders, the composition being formed from the kit of the third or fourth aspects by combining at least the nitrite salt component, the acid component, the conjugate base component (if separate from the nitrite salt component and/or acid component) and aqueous media (e.g. water) (if not present in the other components in sufficient amounts to form the buffer system).

In a sixth aspect, the present invention provides a method of the treatment, amelioration or prevention of respiratory diseases or disorders, wherein the method includes administering a composition or kit of the first to fifth aspects to a subject.

In a seventh aspect, the present invention provides use of a composition or kit of the first to fifth aspects for the manufacture of a medicament for the treatment, amelioration or prevention of respiratory diseases or disorders.

### DESCRIPTION OF THE INVENTION

The present invention will be described in detail with reference to the Examples and accompanying drawings.

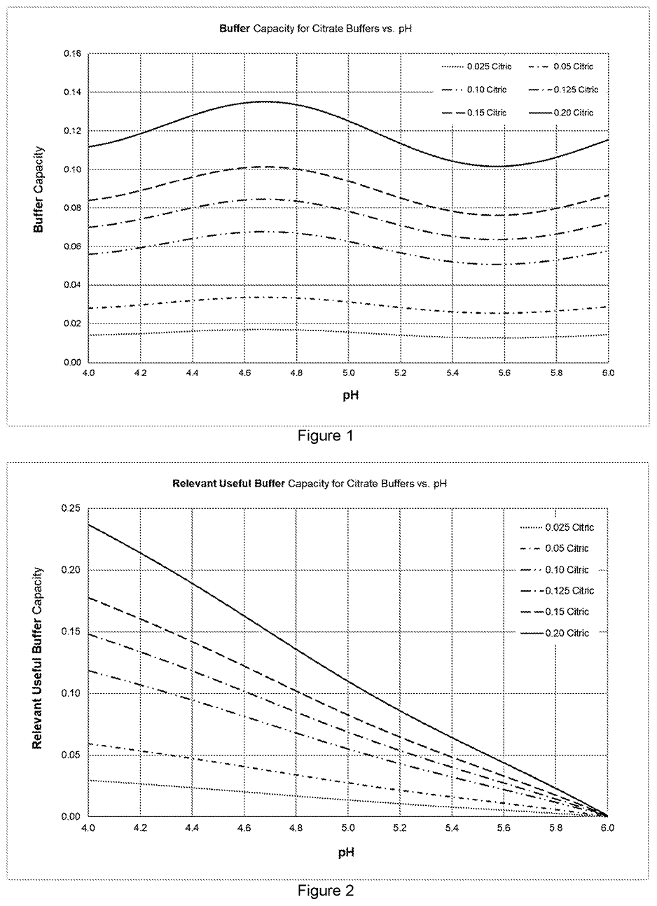

FIG. 1 shows a plot of buffer capacity as calculated by equation (2) as described herein for citric/citrate buffer systems across the pH range of 4.0 to 6.0 at buffer concentrations of 0.025 M, 0.05 M, 0.10 M, 0.125 M, 0.15 M and 0.20 M.

FIG. 2 shows a plot of the relevant useful buffer capacity (as the integral of buffer capacity with respect to pH between the initial pH of the buffer system (pH_i) and a pH of 6.00 using the trapezoidal method at pH intervals of 0.01) for citric/citrate buffer systems across the pH range of 4.0 to 6.0 at buffer concentrations of 0.025 M, 0.05 M, 0.10 M, 0.125 M, 0.15 M and 0.20 M.

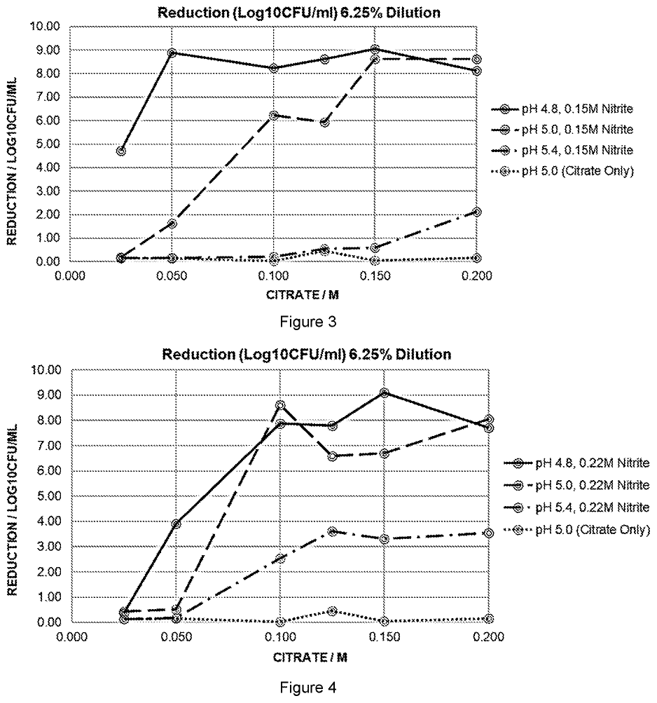

FIG. 3 shows the microbial reduction (Log 10 CFU/mL) of Pseudomonas aeruginosa NCTC 13618 at a 6.25% dilution for formulations including citric/citrate buffers at various concentrations, 0.15 M nitrite, 0.05 M mannitol and pH values of 4.8, 5.0 and 5.4.

FIG. 4 shows the microbial reduction (Log 10 CFU/mL) of Pseudomonas aeruginosa NCTC 13618 at a 6.25% dilution for formulations including citric/citrate buffers at various concentrations, 0.22 M nitrite, 0.05 M mannitol and pH values of 4.8, 5.0 and 5.4.

The reaction between one or more nitrite salt and an acid to generate nitric oxide, optionally other oxides of nitrogen and/or optionally precursors thereof is referred to herein as the “NOx generating reaction” or the “reaction to generate NOx” or like wording, and “NOx” is used to refer to the products of the acidification of nitrite, particularly nitric oxide, other oxides of nitrogen and precursors thereof both individually and collectively in any combination. It will be understood that each component of the generated NOx can be evolved as a gas, or can pass into solution in the reaction mixture, or can initially pass into solution and subsequently be evolved as a gas, or any combination thereof.

The term “target molarity” is used herein to provide the desired molarity of a given component at the point when the components of the nitrite salt, acid and water are first brought together such that a nitric oxide-generating composition is formed.

The term “about” is used herein to denote that the numerical value is not strictly limiting and the skilled person will understand that the value may extend above or below (as appropriate) the exact value in line with the skilled person's understanding of the value. The term “about” may signify a value that is up to ±10% of the value.

Particle size as described herein refers to the volume mean diameter (VMD), unless stated otherwise.

### Buffer System

The present invention includes at least one acid, at least one conjugate base and water that are combined or combinable to form a buffer system. The buffer system has a pH of 4.6 to 6.0, and is further defined either by the buffer capacity or the relevant useful buffer capacity.

### Buffer Capacity

The buffer capacity β of the buffer system may be at least 0.06 as defined by equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of the buffer system;
C_{buf} is the buffer concentration in the nitric oxide-generating composition; and
Kₐ is the dissociation constant of the acid at 25° C.

As explained above, the buffer capacity for acids including polyprotic acids and/or more than one acid and conjugate base pair can be calculated using equation (2), as well as a monoprotic single acid and its conjugate base pair systems. As the pH of a buffer system is not changed by moderate dilution, the pH of the buffer system before addition to the nitrite salt is typically used to provide the [H⁺] value of the buffer system when in the nitric oxide-generating composition. The value of the water dissociation constant at 25° C. is typically taken as 1×10⁻¹⁴.

In some embodiments, the buffer capacity β of the buffer system is at least 0.065, at least 0.070, at least 0.075, at least 0.080, at least 0.085, at least 0.090, at least 0.095 or at least 0.100 as defined by equation (2). Increasing the buffer capacity as defined by equation (2) may improve the effectiveness of the nitric oxide-generating composition.

In some embodiments, the buffer capacity β of the buffer system is up to and including (i.e. no more than) 1.0, up to and including 0.95, up to and including 0.90, up to and including 0.85, up to and including 0.80, up to and including 0.75, up to and including 0.70, up to and including 0.65, up to and including 0.60, or up to and including 0.55 as defined by equation (2).

In particular embodiments, the buffer capacity β of the buffer system is in the range of 0.065 to 0.95, 0.070 to 0.90, 0.075 to 0.85, 0.080 to 0.80, 0.085 to 0.75, 0.090 to 0.70, 0.095 to 0.65 or 0.100 to 0.60 as defined by equation (2). In certain embodiments, the buffer capacity β of the buffer system is in the range of 0.060 to 0.20 as defined by equation (2).

### Relevant Useful Buffer Capacity

The relevant useful buffer capacity Ruβ of the buffer system may be at least 0.04, where the Ruβ is defined as in equation (3)

$$RU β = {\int }_{pH \_ i}^{pH = 6.} β ( pH ) d ( pH ) \qquad ( 3 )$$

as the integral of β with respect to pH between the pH of the buffer system (pH_i) and a pH of 6.00 using the trapezoidal method at pH intervals of 0.01 and β is the buffer capacity of the buffer system as calculated by the equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$

Where:
K_{w} is the water dissociation equilibrium constant at 25° C.;
[H⁺] is the concentration of hydrogen ions, based on the pH of the buffer system;
C_{buf} is the buffer concentration in the nitric-oxide generating composition; and
Kₐ is the dissociation constant of the acid at 25° C.

The considerations for the calculation of the buffer capacity β are the same as above (e.g. where a polyprotic acid or more than one acid/conjugate base pair is used).

The trapezoidal method of calculating integrals is well known. As an example, the integral from 1 to 2 (on the x axis) at 0.5 intervals would be calculated as follows. The value on the y axis at 1 and the value on the y axis at 1.5 (interval of 0.5) is added together and halved in order to get an average y axis value. This average value is then multiplied by the interval, 0.5 to give a first area value. This calculation is repeated for the values on the y axis at 1.5 and 2 to give a second area value. The first and second area values are then added together to give the integral between 1 and 2 using the trapezoidal method at an interval of 0.5. In the present calculation of the Ruβ, this method is used with the pH values on the x-axis and at intervals of pH of 0.01 and the buffer capacity β on the y-axis.

In some embodiments, the relevant useful buffer capacity Ruβ of the buffer system is at least 0.0425, at least 0.045, at least 0.0475, at least 0.050, at least 0.0525, at least 0.055, at least 0.0575 or at least 0.060, where the Ruβ is the integral of β with respect to pH between the pH of the buffer system (pH_i) and a pH of 6.00 using the trapezoidal method at intervals of pH of 0.01 and β is the buffer capacity of the buffer system as calculated by the equation (2).

In some embodiments, the relevant useful buffer capacity Ruβ of the buffer system is at most 0.75, at most 0.70, at most 0.65, at most 0.60, at most 0.55, at most 0.50, at most 0.45, at most 0.40, at most 0.35, at most 0.30, or at most 0.25, where the Ruβ is the integral of β with respect to pH between the pH of the buffer system (pH_i) and a pH of 6.00 using the trapezoidal method at intervals of pH of 0.01 and β is the buffer capacity of the buffer system as calculated by the equation (2).

In particular embodiments, the relevant useful buffer capacity Ruβ of the buffer system is in the range of 0.040 to 0.75 where the Ruβ is the integral of β with respect to pH between the pH of the buffer system (pH_i) and a pH of 6.00 using the trapezoidal method at intervals of pH of 0.01 and β is the buffer capacity of the buffer system as calculated by the equation (2). In other embodiments, the relevant useful buffer capacity Ruβ of the buffer system is in the range of 0.0425 to 0.70, 0.045 to 0.65, 0.050 to 0.60, 0.050 to 0.55, or 0.050 to 0.50 where the Ruβ is the integral of β with respect to pH between the pH of the buffer system (pH_i) and a pH of 6.00 using the trapezoidal method at intervals of pH of 0.01 and β is the buffer capacity of the buffer system as calculated by the equation (2).

### Acids

The compositions and kits include at least one acid. The acid or acids may be involved in the acidification of the nitrite to produce nitric oxide as well as form part of the buffer system. In particular embodiments, the composition or kit include at least one organic acid.

In some embodiments, the composition or kit includes at least one acid having at least one pKa in the range of 4.0 to 6.2 at 25° C. The pKa values of acids are known per se. In certain embodiments, the composition or kit includes at least one organic acid having at least one pKa in the range of 4.0 to 6.2 at 25° C.

In particular embodiments, the acid or acids are one or more organic carboxylic acids or organic non-carboxylic reducing acids.

The expression “organic carboxylic acid” herein refers to any organic acid which contains one or more —COOH group in the molecule. An organic carboxylic acid may be straight-chain or branched. The carboxylic acid may be saturated or unsaturated. The carboxylic acid may be aliphatic or aromatic. The carboxylic acid may be acyclic or cyclic. The carboxylic acid may be a vinylogous carboxylic acid.

The organic carboxylic acid may carry one or more substituents, for example one or more hydroxyl group. Examples of hydroxyl-substituted organic carboxylic acids which may be used in the present disclosure include α-hydroxy-carboxylic acids, p-hydroxy-carboxylic acids and γ-hydroxy-carboxylic acids.

The expression “organic non-carboxylic reducing acid” herein refers to any organic reducing acid which does not contain a —COOH group in the molecule. An organic non-carboxylic reducing acid may be straight-chain or branched. The non-carboxylic reducing acid may be saturated or unsaturated. The non-carboxylic reducing acid may be aliphatic or aromatic. The non-carboxylic reducing acid may be acyclic or cyclic. The non-carboxylic reducing acid may be vinylogous.

The organic non-carboxylic reducing acid may carry one or more substituents, for example one or more hydroxyl group. Examples of hydroxyl-substituted organic non-carboxylic reducing acids which may be used in the present disclosure include the acidic reductones, for example reductic acid (2.3-dihydroxy-2-cyclopentanone).

The one or more organic carboxylic acid may comprise, consist of, or be one or more reducing carboxylic acids. The organic carboxylic acid may, for example, be selected from salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, p-hydroxypropanoic acid, p-hydroxybutyric acid, p-hydroxy-β-butyric acid, naphthoic acid, oleic acid, palmitic acid, pamoic (emboic) acid, stearic acid, malonic acid, succinic acid, fumaric acid, glucoheptonic acid, glucuronic acid, lactobioic acid, cinnamic acid, pyruvic acid, orotic acid, glyceric acid, glycyrrhizic acid, sorbic acid, hyaluronic acid, alginic acid, oxalic acid, salts thereof, and combinations thereof.

The organic carboxylic acid may be citric acid or a salt thereof.

The carboxylic acid may be or comprise a polymeric or polymerised carboxylic acid such as, for example, polyacrylic acid, polymethacrylic acid, a copolymer of acrylic acid and methacrylic acid, polylactic acid, polyglycolic acid, or a copolymer of lactic acid and glycolic acid. The term “organic carboxylic acid” used herein also cover partial or full esters of organic carboxylic acids or partial or full salts thereof, provided that those can serve as an acid in use according to the present invention.

The organic non-carboxylic reducing acid may, for example, be selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid, 6-O-dodecanedioyl ascorbic acid, L-Ascorbic acid 2-phosphate and 2-O-alpha-D-Glucopyranosyl-L-ascorbic acid; acidic reductones such as reductic acid; erythorbic acid; salts thereof; and combinations thereof.

The organic non-carboxylic reducing acid may be ascorbic acid or a salt thereof.

In certain embodiments, the composition or kit includes at least one organic carboxylic acid having at least one pKa in the range of 4.0 to 6.2 at 25° C.

In some embodiments, the composition or kit described herein may further include an additional acid suitable for the acidification of nitrite.

### pH of the Buffer System

The acid and its conjugate base may be provided in a ratio to achieve the desired pH in the buffer system. The buffer system has a pH between 4.6 and 6.0. In some embodiments, the buffer system has a pH between 4.6 and 5.6, between 4.6 and 5.4, between 4.6 and 5.2 or between 4.6 and 5.0. In alternative embodiments, the buffer system has a pH between 4.8 and 5.2. Typically, the pH of the buffer system can be determined prior to addition of the nitrite salt to form the composition.

The conjugate base may be added separately, or may be generated in situ from the acid by adjustment of the pH using an acid and/or base, for example a mineral acid and/or a mineral base.

### Buffer Concentration

Buffer concentration is dependent on the required pH and required buffer capacity or relevant useful buffer capacity, and can be determined from the above equation and calculations.

For the purposes of the above calculations, the buffer concentration is the concentration in the buffer system in the nitric oxide-generating composition immediately after forming the composition (e.g. immediately after mixing components such as the acid and nitrite salt components). The composition may have a buffer concentration in the range of 0.01 M to 1.5 M, 0.05 M to 1.0 M, 0.075 M to 0.75 M. In particular embodiments, the compositions have a buffer concentration of at least 0.075 M, at least 0.10 M, at least 0.11 M, at least 0.12 M, at least 0.13 M, at least 0.14 M or at least 0.15 M. In particular embodiments, compositions have a buffer concentration of at most 0.75 M, at most 0.70 M, at most 0.65 M, at most 0.60 M, at most 0.55 M or at most 0.50 M.

The kits as described herein typically include the acid and conjugate base in amounts that provide the required buffer concentration when combined with other components to form the nitric oxide-generating composition. The kit may include the acid and conjugate base in amounts that provide a target buffer concentration in the range of 0.01 M to 1.5 M, 0.05 M to 1.0 M, 0.075 M to 0.75 M when combined with other components to form the nitric oxide-generating composition. In particular embodiments, the kits include the acid and conjugate base in amounts required to provide a target buffer concentration of at least 0.075 M, at least 0.10 M, at least 0.11 M, at least 0.12 M, at least 0.13 M, at least 0.14 M or at least 0.15 M when combined with other components to form the nitric oxide-generating composition. In particular embodiments, the kits include the acid and conjugate base in amounts required to provide a target buffer concentration of at most 0.75 M, at most 0.70 M, at most 0.65 M, at most 0.60 M, at most 0.55 M or at most 0.50 M when combined with other components to form the nitric oxide-generating composition.

It should be appreciated that an aqueous solution of the acid and an aqueous solution of nitrite salt will be diluted when the two separate solutions are combined to form a composition. As such, the molarity of an aqueous solution of the acid and the molarity an aqueous solution of nitrite salt will be higher than the overall desired (or target) molarity on mixing of each component to take into account the dilution.

### Nitrite Salt/Nitrite Salt Component

The present invention includes one or more nitrite salts. The choice of nitrite salt is not particularly limited. The nitrite salt may be selected from one or more alkali metal nitrite salts or alkaline metal nitrite salts. For example, the one or more nitrite salt may be selected from LiNO₂, NaNO₂, KNO₂, RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof. The nitrite salt may be NaNO₂ or KNO₂. The nitrite salt may be NaNO₂.

The nitrite salt may be a pharmaceutically acceptable grade of nitrite salt. In other words, the nitrite salt may adhere to one or more active pharmacopoeia monographs for the nitrite salt. For example, the nitrite salt may adhere to the monograph of the nitrite salt of one or more of the United States Pharmacopoeia (USP), European Pharmacopoeia or Japanese Pharmacopoeia.

In particular, the nitrite salt used may have one or more of the characteristics as provided in paragraphs [0032] to [0060] and/or Table 1 in paragraph [0204] of WO 2010/093746, the disclosure of which is incorporated herein by reference in its entirety.

### Molarity of Nitrite Salt

The molarity of the nitrite salt in the composition may be in the range of 0.001 M to 2.0 M. In some embodiments, the molarity of the nitrite salt in the composition is in the range of 0.01 M to 1.5 M, 0.05 M to 1.0 M, 0.075 M to 0.75 M. In particular embodiments, the molarity of the nitrite salt in the composition is at least 0.075 M, at least 0.080 M, at least 0.085 M, at least 0.090 M, at least 0.095 M or at least 0.100 M. In particular embodiments, the molarity of the nitrite salt in the composition is at most 0.75 M, at most 0.70 M, at most 0.65 M, at most 0.60 M, at most 0.55 M or at most 0.50 M.

In some embodiments, the ratio of the molarity of the nitrite salt to the buffer concentration is about 1:1 in the composition. In other embodiments, the ratio of the molarity of the nitrite salt to the buffer concentration is greater than 1:1 in the composition.

The target molarity (i.e. the molarity after mixing of the acid and nitrite salt components) of the nitrite salt in the kit may be in the range of 0.001 M to 2.0 M. In some embodiments, the target molarity of the nitrite salt in the kit is in the range of 0.01 M to 1.5 M, 0.05 M to 1.0 M, 0.075 M to 0.75 M. In particular embodiments, the target molarity of the nitrite salt in the kit is at least 0.075 M, at least 0.080 M, at least 0.085 M, at least 0.090 M, at least 0.095 M or at least 0.100 M. In particular embodiments, the target molarity of the nitrite salt in the kit is at most 0.75 M, at most 0.70 M, at most 0.65 M, at most 0.60 M, at most 0.55 M or at most 0.50 M.

In some embodiments, the ratio of the target molarity of the nitrite salt to the target buffer concentration is about 1:1 in the kit. In other embodiments, the ratio of the target molarity of the nitrite salt to the target acid is greater than 1:1 in the kit.

### Form of the Components of the Kit

Acid and/or Nitrite Salt in Aqueous Solution

In some embodiments, one or both of the acid component and the nitrite salt component are in an aqueous solution. In these embodiments, the acid component and nitrite salt component are typically kept as separate components until the point of need so as to prevent acidification of the nitrite salt before required.

In a particular embodiment, the acid component and the nitrite salt are both in aqueous solution. In this embodiments, the conjugate base may be included in the acid component. In this way, the aqueous acid component may form the buffer system.

Acid and/or Nitrite Salt in Solid Form

In other embodiments, the kit includes the acid component and/or the nitrite salt component in solid form. In these embodiments, the kit further includes instructions with the volume of aqueous media (e.g. water) required to be added in order to form the nitric oxide generating composition as described herein. In some embodiments, the kit includes a separate container with a fixed volume of aqueous media and instructions to add the fixed volume of aqueous media to generate the buffer system.

In some embodiments, the kit includes a solid powder composition as described in PCT/GB2022/53305 or PCT/GB2022/53307, the content of which are incorporated herein by reference. The solid powder composition described therein typically includes both acid and nitrite salt held together in proximity to allow effective storage until usage.

The solid powder composition may include particles that are:
(a) Particles containing both a nitrite salt and an acid; and/or
(b) An agglomeration of particles, wherein the agglomeration of particles includes one or more nitrite particles containing a nitrite salt and one or more acid particles containing an acid.

In this way, the acid component and nitrite salt component may be held in close proximity. It is to be understood that the particles may contain the nitrite salt and the acid within the same particle when the particles contain both the acid and the nitrite salt.

The expressions “agglomerate”, “agglomeration” and “agglomerated together” herein refer to an aggregation or assemblage of primary particles exhibiting an identifiable collective behaviour. In the present invention, the primary particles may be nitrite particles containing a nitrite salt, acid particles containing an acid, or particles containing both a nitrite salt and an acid. In the present invention, an identifiable collective behaviour may be resistance to mechanical separation, i.e., the particles adhesion to one another.

The particles or agglomerates of the solid composition may be a suitable particle size for their desired use or application. For example, the particles or agglomerates of the solid composition may have a particle size of about 10 μm or less, for example, about 5 μm or less, about 4 μm or less, about 3 μm or less, about 2 μm or less or about 1 μm or less.

Alternatively, the particles or agglomerates of the solid composition may have a particle size of greater than 5 μm. For example, the particles or agglomerates of the solid composition may have a particle size of greater than 50 μm, greater than 100 μm, greater than 250 μm, greater than 500 μm, greater than 750 μm, greater than 1000 μm.

The weight ratio of nitrite to acid in the solid composition may be in the range of about 1:1 to about 1:99, such as in the range of about 1:4 to about 1:49 or about 1:7 to about 1:24.

The solid powder composition may be substantially free of one or more binding agents. Alternatively, the solid powder composition may further include one or more binding agents. A “binding agent” used herein refers to an agent that promotes the adhesion of particles, i.e. promotes the formation of an agglomeration of particles.

Suitable binding agents may include sugars, natural binders or synthetic or semisynthetic polymer binders. Sugar species may include, for example, sucrose or liquid glucose. Natural binders may include, for example, acacia, tragacanth, gelatin, starch paste, pregelatinized starch, alginic acid or cellulose. Synthetic or semisynthetic polymer binders may include, for example, methyl cellulose, ethyl cellulose, hydroxy propyl methyl cellulose (HPMC), hydroxy propyl cellulose, sodium carboxy methyl cellulose, polyvinylpyrrolidones (PVP), polyethylene glycols (PEG), polyvinyl alcohols, polymethacrylates. The binding agent may be a copolymer of 1-vinyl-2-pyrrolidone and vinyl acetate (copovidone). The binding agent may be microcrystalline cellulose.

The binding agent may be incorporated into the composition in % w/w of about 5% w/w to about 30% w/w. For example, the binding agent may be incorporated into the composition in a % w/w of about 10% w/w to about 25% w/w.

The agglomeration of particles may be achieved by any suitable means, known to the person of skill in the art.

The agglomeration of particles may be achieved by mechanical means, for example, by mechanically forcing the particles together. Agglomeration by mechanical means may be achieved by micronizing particles of a nitrite salt and particles of an acid. Alternatively, agglomeration by mechanical means may be achieved by having particles that are substantially static-free.

The agglomeration of particles may be achieved by chemical means, for example, chemically facilitated adhesion or a chemical coating. Agglomeration by chemical means may be achieved by adhesion promoters, for example, moisture. Alternatively, agglomeration by chemical means may be achieved by a coating material that binds primary particles of a nitrite salt and primary particles of an acid together. Suitable binding agents are previously discussed, and suitable coating materials are discussed in the section “Coated particles” below.

The solid powder composition may include particles coated with a hydrophobic material (also referred to herein as coated particles).

The coated particles may include a single particle containing a nitrite salt and an acid and coated with the hydrophobic material.

Alternatively, the coated particles may be an agglomeration of particles coated with the hydrophobic material and the agglomeration of particles includes (a) particles containing a nitrite salt and an acid and/or (b) a mixture of one or more nitrite salt particles containing a nitrite salt and one or more acid particles containing an acid.

In this way, the coated particles include nitrite salt and acid within the same coating.

The hydrophobic material may be any material capable of coating the particles or agglomerates such that the particles or agglomerates are coated with a hydrophobic layer. The hydrophobic material may be a polymeric material, for example an organic polymeric material. The hydrophobic material may be an amphiphilic species, for example, a surfactant-type species such as a non-ionic, anionic, cationic or amphoteric surfactant-type species. The hydrophobic material may be an inorganic mineral material, for example, and inorganic mineral material that forms a 3D framework. The hydrophobic material may be biocompatible. The hydrophobic material may include one or more of poly(lactic-co-glycolic acid) (PLGA), dipalmitoylphosphatidylcholine (DPPC), magnesium stearate, and mesoporous silica. The hydrophobic material may comprise the polymeric material poly(lactic-co-glycolic acid) (PLGA) without an acid end group or may comprise the polymeric material poly(lactic-co-glycolic acid) (PLGA) with an acid end group.

A “surfactant” as used herein refers to a surface-active agent which can lower the surface tension of a species in a medium or the interfacial tension between mediums. Surfactant species generally have a hydrophilic head and a hydrophobic tail.

The hydrophobic material may adhere to the particles or agglomerates by chemical bonding or by electrostatic or intermolecular forces.

The coating of the coated particles or coated agglomeration of particles may affect the reaction dynamics, for example the reaction kinetics, of the acidification of the nitrite salt when the coated particles or coated agglomerates are exposed to an aqueous environment.

The coated particles or coated agglomeration of particles of the solid composition may be a suitable particle size for the desired use or application. The coated particles or coated agglomeration of particles of the solid composition may have a particle size of about 10 μm or less, for example, about 5 μm or less, about 4 μm or less, about 3 μm or less, about 2 μm or less or about 1 μm or less. Alternatively, the coated particles or coated agglomerates of the solid composition may have a particle size of greater than about 5 μm. For example, the particles or agglomerates of the solid composition may have a particle size of greater than about 50 μm, greater than about 100 μm, greater than about 250 μm, greater than about 500 μm, greater than about 750 μm, greater than about 1000 μm.

Forming such a solid powder composition may be performed in a number of ways.

Forming the Particles from a Mixture Containing a Nitrite Salt Solution and an Acid Solution

The particles of the solid powder composition may be formed from a mixture containing a nitrite salt solution and an acid solution. Particles formed in this way should be formed by removal of solvent in a short time (e.g., thirty seconds or less) after mixing the nitrite salt solution and the acid solution and/or the mixture is placed under reaction retarding conditions (e.g. at a temperature less than the freezing point of the solvent) after mixing nitrite salt solution and the acid solution and for solvent removal. In this way, the solvent is removed from the mixture while minimising the acidification of the nitrite. An effective amount of nitrite and acid may therefore be present in the resulting powder composition.

When the solvent is removed in a short time after mixing the nitrite salt solution and the acid solution, the solvent may be removed in thirty second or less after the nitrite solution and acid solution is mixed. In some examples, the solvent is removed in ten seconds or less, five seconds or less, two seconds or less or one second or less after mixing the nitrite solution and the acid solution. In some examples, the solvent is removed in 500 milliseconds or less, 100 milliseconds or less, 50 milliseconds or less or 10 milliseconds or less after mixing the nitrite solution and the acid solution.

In one example, the particles may be formed by spray-drying a mixture containing a nitrite salt solution and an acid solution. Spray-drying of the mixture may allow the removal of solvent in a time of thirty seconds or less after mixing of the nitrite salt solution and the acid solution. Spray-drying of materials is known per se.

The mixture is typically a mixture of an aqueous solution of the nitrite salt and an aqueous solution of the acid. When aqueous solutions are used, the time between mixing the two aqueous solutions is minimised to suppress acidification of the nitrite salt. The aqueous solution of the nitrite salt and the aqueous solution of the acid may be mixed in line for about 1 to about 10 milliseconds, for example about 3 to about 5 milliseconds, before spray-drying takes place. Spray-drying may occur immediately after mixing of the nitrite and acid solutions. It is understood that mixing and spray-drying a mixture containing a nitrite salt solution and an acid solution, as described, limits the potential reaction time between the acid and nitrite component.

The particles formed by spray-drying the mixture containing a nitrite salt solution and an acid solution may have a particle size of about 10 μm or less, for example, about 5 μm or less, about 4 μm or less, about 3 μm or less, about 2 μm or less, or about 1 μm or less.

Spray-drying a mixture containing a nitrite salt solution and an acid solution as described may result in a solid powder composition where each particle contains nitrite salt and acid components.

Particles formed by spray-drying a mixture containing a nitrite salt solution and an acid solution may be any suitable morphology. For example, particles formed by spray-drying a mixture containing a nitrite salt solution and acid solution may be crystalline in form or amorphous in form. The particles formed by spray-drying a mixture containing a nitrite salt solution and an acid solution may be amorphous in form.

Additionally or alternatively, the mixture of nitrite salt solution and acid solution is placed under a reaction-retarding condition (e.g. at a temperature less than the freezing point of the solvent) before, during or immediately after mixing the nitrite salt solution and the acid solution and for solvent removal. In this way, the acidification of the nitrite is retarded until the solvent is removed. In particular, the solvent may be an aqueous solvent.

A particular example of a reaction-retarding condition is a temperature of the mixture below the freezing point of the solvent. In this way, the reaction rate of the acidification of nitrite may be slowed while the solvent is removed. Where the temperature of the mixture is below the freezing point of the solvent, the nitrite solution and the acid solution are typically mixed at a temperature above the freezing point of the solvent before the temperature of the mixture is reduced to below the freezing point of the solvent. In this way, good mixing of the solutions may occur.

In some examples, the solvent removal may occur at a reduced gas pressure. In particular, the solvent removal may occur at a reduced gas pressure in combination at a temperature below the freezing point of the solvent to be removed.

A particularly useful technique to remove the solvent under a reaction-retarding condition is lyophilisation (also referred to as “freeze-drying”).

It should be noted that the terms “removal of solvent” and/or “drying” as used herein to achieve a solid powder composition. These terms include but are not limited to the complete removal of solvent. In some examples, a solid powder composition may include trace amounts of residual solvent. For example, the powder composition may contain up to about 10% of residual solvent, for example up to about 5% residual solvent, up to about 3% residual solvent or up to about 1% residual solvent. Additional drying techniques, such as vacuum drying, may be employed after the initial removal of solvent in order to provide the solid powder composition.

### Combining Solids to Form an Agglomeration of Particles

The solid powder composition may be formed by combining a nitrite-containing solid with an acid-containing solid to form an agglomeration of particles, wherein the agglomeration of particles includes one or more particles containing a nitrite salt and one or more particles containing an acid.

Combining a nitrite-containing solid with an acid-containing solid to form an agglomeration of particle may be achieved, for example, by (a) blending one or more nitrite salt particles and one or more acid particles, wherein the nitrite salt particles are formed by spray-drying a nitrite salt solution and the acid particles are formed by spray-drying acid solution; or (b) forming one or more particles by micronizing a nitrite salt solid with an acid solid.

### Blended Spray-Dried Nitrite Particles and Spray-Dried Acid Particles

The solid powder composition may be a blend of nitrite salt particles and acid particles, wherein the nitrite salt particles are formed by spray-drying a nitrite salt solution and the acid particles are formed by spray-drying an acid solution. The spray-dried nitrite salt particles and the spray-dried acid particles may be blended by standard means known to a person of skill in the art to provide a blended solid powder composition.

The spray-dried nitrite particles and the spray-dried acid particles may be blended at a nitrite to acid weight ratio of about 1:1 to about 1:99, such as in the range of about 1:4 to about 1:49 or about 1:7 to about 1:24.

The spray-dried particles of nitrite salt and the spray-dried particles of acid may be blended for a time of, about 5 to about 60 minutes, for example a time of about 10 to about 40 minutes, or a time of about 15 to about 30 minutes. The spray-dried particles of nitrite salt and the spray-dried particles of acid may be blended for a time of about 20 minutes.

The particles formed by a nitrite salt solution and spray-drying an acid solution and blending these components as described may have a particle size of about 10 μm or less, for example, about 5 μm or less, about 4 μm or less, about 3 μm or less, about 2 μm or less, or about 1 μm or less.

Spray-drying a nitrite salt solution and spray-drying an acid solution and blending these components as described may result in a solid powder composition including an agglomeration of particles, wherein the agglomeration includes one or more particles containing nitrite salt and one or more particles containing acid.

Particles formed by spray-drying a nitrite salt solution and spray-drying an acid solution and blending these components may be any suitable morphology. For example, particles formed by spray-drying a nitrite salt solution and spray-drying acid solution and blending these components may be crystalline in form or amorphous in form. The particles formed by spray-drying a mixture containing a nitrite salt solution and acid solution may be amorphous in form.

Particles Formed from Micronizing a Nitrite Salt Solid with an Acid Solid

The particles may be formed by micronizing a nitrite salt solid with an acid solid.

The expression “micronizing” as used herein refers to a process for reducing the average particle size of a solid composition, typically to within the micrometre scale. Micronizing can be achieved by standard processes known to a person of skill in the art. For example, micronizing may occur by milling or grinding the particles or by utilisation of super critical fluids.

Where the acid is a buffered acid system, the acid solid may be two components, a solid acid component and a solid conjugate base component. The nitrite salt solid and the acid solid may be micronized in a ratio of about 1:1 to about 1:99, such as in the range of about 1:4 to about 1:49 or about 1:7 to about 1:24, e.g. 1:9 w/w nitrite:acid.

The particles formed by micronizing a nitrite salt solid with an acid solid may have a particle size of about 10 μm or less, for example, about 5 μm or less, about 4 μm or less, about 3 μm or less, about 2 μm or less, or about 1 μm or less.

Micronizing a nitrite salt solution with an acid solution as described may result in a solid powder composition of particles containing nitrite salt and particles containing acid. Micronizing a nitrite salt solution with an acid solution as described may result in a solid powder composition which comprises agglomerates comprising particles containing nitrite salt and particles containing acid.

Particles formed by micronizing a nitrite salt solution with an acid solution may be any suitable morphology. For example, particles formed by micronizing a nitrite salt solution with an acid solution may be crystalline in form or amorphous in form. The particles formed by micronizing a nitrite salt solution with an acid solution may be crystalline in form.

The particles formed by micronizing may include one or more of the optional additives (in addition to the acid and nitrite salt) as described above. In particular, the particles formed by micronizing may include a binding agent as described above. The binding agent may be micronized with the nitrite solid and the acid solid.

### Optional Components

The composition and kits of the present invention may include one or more optional components.

### Organic Polyol

The composition or kit of the present invention may be substantially free of one or more organic polyols.

Alternatively, the composition or kit of the present invention may further include one or more organic polyol. The expression “organic polyol” herein refers to an organic molecule with two or more hydroxyl groups that is not an acid, particularly for a nitrite salt reaction, and is not a saccharide or polysaccharide (the terms “saccharide” and “polysaccharide” include oligosaccharide, glycan and glycosaminoglycan). The organic polyol will thus have a pKa₁ of about 7 or greater.

The expression “organic polyol” herein preferably excludes reductants. Examples of reductants which are organic molecules with two or more hydroxyl groups and not a saccharide or polysaccharide are thioglycerol (for example, 1-thioglycerol), hydroquinone, butylated hydroquinone, ascorbic acid, ascorbate, erythorbic acid and erythorbate. Thioglycerol (for example, 1-thioglycerol), hydroquinone, butylated hydroquinone, ascorbate and erythorbate are thus preferably excluded from the expression “organic polyol” because they are reductants. Ascorbic acid and erythorbic acid are excluded from the expression anyway because they are acids, particularly for the nitrite salt reaction.

The organic polyol may be cyclic or acyclic or may be a mixture of one or more cyclic organic polyol and one or more acyclic organic polyol. For example, the one or more organic polyol may be selected from one or more alkane substituted by two or more OH groups, one or more cycloalkane substituted by two or more OH groups, one or more cycloalkylalkane substituted by two or more OH groups, and any combination thereof. The organic polyol may not carry any substituents other than OH.

The one or more organic polyol may be one or more acyclic organic polyol. The one or more acyclic organic polyol may be selected from the sugar alcohols having 4, 5, 6, 7, 8, 9, 10, 11 or 12 carbon atoms. The one or more acyclic organic polyol may be selected from the alditols, for example the alditols having 4, 5, 6, 7, 8, 9, 10, 11 or 12 carbon atoms. The one or more organic polyol may not include a saponin, sapogenin, steroid or steroidal glycoside.

Alternatively, the one or more organic polyol may be one or more cyclic organic polyol. The one or more cyclic organic polyol may be a cyclic sugar alcohol or a cyclic alditol. For example, the one or more cyclic polyol may be a cyclic sugar alcohol having 4, 5, 6, 7, 8, 9, 10, 11 or 12 carbon atoms or a cyclic alditol having 4, 5, 6, 7, 8, 9, 10, 11 or 12 carbon atoms. A specific example of a cyclic polyol is inositol.

The one or more organic polyol may have 7 or more hydroxy groups. The one or more organic polyol may be a sugar alcohol or alditol having 7 or more hydroxy groups. The one or more organic polyol may have 9 or more hydroxy groups. The one or more organic polyol may be a sugar alcohol or alditol having 9 or more hydroxy groups. The one or more organic polyol may have 20 or fewer hydroxyl groups. The one or more organic polyol may be a sugar alcohol or alditol having 20 or fewer hydroxy groups. The one or more organic polyol may have 15 or fewer hydroxyl groups. The one or more organic polyol may be a sugar alcohol or alditol having 15 or fewer hydroxyl groups. The one or more organic polyol may have a number of hydroxyl groups in the range of 7 to 20, for example, in the range of 9 to 15. The one or more organic polyol may include 9, 12, 15 or 18 hydroxy groups.

The one or more organic polyol may be a sugar alcohol compound comprising, for example consisting of one or more monosaccharide units and one or more acyclic sugar alcohol units. The one or more organic polyol may be a sugar alcohol compound comprising, for example consisting of, a straight chain of one or more monosaccharide units and one or more acyclic sugar alcohol units or a branched chain of one or more monosaccharide units and one or more acyclic sugar alcohol units.

A “monosaccharide unit” as used herein refers to a monosaccharide covalently linked to at least one other unit (whether another monosaccharide unit or an acyclic sugar alcohol unit) in the compound. An “acyclic sugar alcohol unit” as used herein refers to an acyclic sugar alcohol linked covalently to least one other unit (whether a monosaccharide unit or another acyclic sugar alcohol unit) in the compound. The units in the compound may be linked through ether linkages. One or more of the monosaccharide units may be covalently linked to other units of the compound through a glycosidic bond. Each of the monosaccharide units may be covalently linked to other units of the compound through a glycosidic bond. The sugar alcohol compound may be a glycoside with a monosaccharide or oligosaccharide glycone and an acyclic sugar alcohol aglycone.

Acyclic sugar alcohol units may be sugar alcohol units having 4, 5, 6, 7, 8, 9, 10, 11 or 12 carbon atoms. The acyclic sugar alcohol unit may be selected from the group consisting of units of erythritol, threitol, arabitol, xylitol, ribitol, mannitol, sorbitol, galactitol, fucitol, iditol and volemitol.

One or more of the monosaccharide units may be a C₅ or C₆ monosaccharide unit, i.e., a pentose or hexose unit. Each monosaccharide unit may be a C₅ or C₆ monosaccharide unit. One or more of the sugar alcohol units may be a C₅ or C₆ sugar alcohol unit. Each sugar alcohol unit may be a C₅ or C₆ sugar alcohol unit.

The sugar alcohol compound may comprise, for example may consist of, n monosaccharide units and m acyclic sugar alcohol units, where n is a whole number and at least one, m is a whole number and at least one and (n+m) is no more than 10. The sugar alcohol compound may comprise, for example may consist of, a chain of n monosaccharide units terminated with one acyclic sugar alcohol unit, where n is a whole number between one and nine. The chain of monosaccharide units may be covalently linked by glycosidic bonds. Each monosaccharide unit may be covalently linked to another monosaccharide unit or the acyclic sugar alcohol unit by a glycosidic bond. The sugar alcohol compound may comprise, for example may consist of, a chain of 1, 2 or 3 monosaccharide units terminated with one acyclic alcohol unit. 1, 2, 3 or each monosaccharide unit may be a C₅ or C₆ monosaccharide unit. The acyclic alcohol unit may be a C₅ or C₆ sugar alcohol unit. Examples of the sugar alcohol compound include but are not limited to: isomalt, maltitol and lactitol (n=1); maltotriitol (n=2); and maltotetraitol (n=3).

Such sugar alcohol compounds may be described as sugar alcohols derived from a disaccharide or an oligosaccharide. “Oligosaccharide”, as used herein, refers to a saccharide consisting of three to ten monosaccharide units. Sugar alcohols derived from disaccharides or oligosaccharides may be synthesised (e.g. by hydrogenation) from disaccharides, oligosaccharides or polysaccharides (e.g. from hydrolysis and hydrogenation), but are not limited to compounds synthesised from disaccharides, oligosaccharides or polysaccharides. For example, sugar alcohols derived from a disaccharide may be formed from the dehydration reaction of a monosaccharide and a sugar alcohol. The one or more organic polyol may be a sugar alcohol derived from a disaccharide, trisaccharide or tetrasaccharide. Examples of sugar alcohols derived from disaccharides include but are not limited to isomalt, maltitol and lactitol. An example of a sugar alcohol derived from a trisaccharide includes but is not limited to maltotriitol. An example of a sugar alcohol derived from a tetrasaccharide includes but is not limited to maltotetraitol.

Organic polyols may be selected from erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof. Glycerol can be used, and when present is preferably in association with one or more other organic polyol, for example erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof.

Many organic polyols contain one or more chiral centre and thus exist in stereoisomeric forms. All stereoisomeric forms and optical isomers and isomer mixtures of the organic polyols are intended to be included within the scope of this invention. In particular, the D and/or L forms of all chiral organic polyols and all mixtures thereof may be used.

When the composition or kit is in solid form and includes one or more organic polyols, it is preferred that the organic polyol is added to the composition after any processing which involves removal of solvent (e.g., after spray drying or lyophilisation steps). In other words, the polyol may be added to a composition including one or more particles containing a nitrite salt and an acid; or added to a composition including one or more particles containing a nitrite salt and/or one or more particles containing an acid (either before or after an agglomeration of these particles is formed).

### Molarity of Organic Polyol

The molarity of the organic polyol in the composition may be in the range of 0.001 M to 1.0 M. In some embodiments, the molarity of the organic polyol in the composition is in the range of 0.002 M to 0.5 M, 0.003 M to 0.1 M, 0.005 M to 0.05 M. In particular embodiments, the molarity of the organic polyol in the composition is at least 0.005 M, at least 0.008 M, at least 0.01 M, or at least 0.012 M. In particular embodiments, the molarity of the nitrite salt in the composition is at most 0.05 M, at most 0.045 M, at most 0.035 M, or at most 0.025 M.

### Certain Embodiments

In certain embodiments, the kit includes the nitrite salt component as an aqueous solution comprising one or more nitrite salts and the acid component as an aqueous solution including an acid comprising one or more organic carboxylic acids and organic non-carboxylic reducing acids.

In certain embodiments, the buffer capacity β of buffer system is in the range of 0.060 to 0.20 as defined by equation (2). In certain embodiments, the relevant useful buffer capacity Ruβ of the buffer system is in the range of 0.040 to 0.75 where the Ruβ is the integral of β with respect to pH between the pH of the buffer system (pH_i) and a pH of 6.00 using the trapezoidal method at intervals of pH of 0.01 and β is the buffer capacity of the buffer system as calculated by the equation (2).

In some embodiments, the buffer system is selected so that a pH between 4.8 and 5.2 or between 4.6 and 5.0 is achieved upon exposure to an aqueous environment.

In some embodiments, the buffer system includes citric acid and citrate salt as the acid and conjugate base. In these embodiments, the target buffer concentration may be in the range of 0.025 M to 0.25 M (depending on the pH of the acid). The target molarity of the nitrite salt may be in the range of 0.025 M to 0.25 M. In some embodiments, the ratio of the target molarity of the nitrite salt to the target buffer concentration may be in the range of about 0.8:1 to about 2:1. In some embodiments, the ratio of the target molarity of the nitrite salt to the target buffer concentration may be greater than 1:1. In other words, the target molarity of the nitrite salt may be greater than the target buffer concentration. The target molarity of the nitrite salt may be greater than the target buffer concentration when the pH of the buffer is greater than 5.2.

The composition or kit may further include an organic polyol selected from the group consisting of erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof. Such organic polyol may be in either the nitrite salt component or the acid component. In certain embodiments, the composition or kit includes mannitol.

### Pharmaceutical Compositions

The composition or one or more components of the kit disclosed herein may be included in a pharmaceutical composition, optionally with one or more pharmaceutically acceptable carriers, excipients and/or adjuvants. Such carriers, excipients and/or adjuvants may be physiologically compatible when desired for use in vivo.

Examples of carriers and/or excipients, for example carriers and or excipients that are physiologically compatible, include without limitation lactose, starch, dicalcium phosphate, magnesium stearate, sodium saccharin, talcum, cellulose, cellulose derivatives, sodium croscarmellose, glucose, gelatin, sucrose, magnesium carbonate, magnesium chloride, magnesium sulfate, calcium chloride and the like.

Generally speaking, depending on the intended mode of administration, the pharmaceutical composition will contain about 0.005% to about 95%, preferably about 0.5% to about 50% by weight of the combination or composition of the present invention or components thereof. Actual methods of preparing such dosage forms are known, or will be apparent to those skilled in the art.

Excipients may be selected from known excipients depending on the intended use or administration route whereby the reactants and/or reaction products are to be delivered to the target site for the delivery of the nitric oxide. Optional additional components may, for example, be selected from sweetening agents, taste-masking agents, wetting agents, lubricants, binders, emulsifiers, solubilising agents, stabilising agents, colourants, odourants, salts, coating agents, antioxidants, pharmaceutically active agents and preservatives. Such components are well known in the art and a detailed discussion of them is not necessary for the skilled reader. Examples of auxiliary substances such as wetting agents, emulsifying agents, lubricants, binders, and solubilising agents include, for example, sodium phosphate, potassium phosphate, gum acacia, polyvinylpyrrolidone, cyclodextrin derivatives, sorbitan monolaurate, triethanolamine acetate, triethanolamine oleate and the like. A sweetening agent or a taste-masking agent may, for example, include a sugar, saccharin, aspartame, sucralose, neotame or other compound that beneficially affects taste, after-taste, perceived unpleasant saltiness, sourness or bitterness, that reduces the tendency of an oral or inhaled formulation to irritate a recipient (e.g. by causing coughing or sore throat or other undesired side effect, such as may reduce the delivered dose or adversely affect patient compliance with a prescribed therapeutic regimen). Certain taste-masking agents may form complexes with one or more of the nitrite salts.

Examples of pharmaceutically active agents that may be incorporated in the components and compositions or co-administered with the components and compositions according to the present invention include antibiotics, steroids, anaesthetics (for example topical anaesthetics such as lignocaine (lidocaine), amethocaine (tetracaine), xylocaine, bupivacaine, prilocaine, ropivfacaine, benzocaine, mepivocaine, cocaine or any combination thereof), analgesics, anti-inflammatory agents (for example non-steroidal anti-inflammatory drugs (NSAIDs)), anti-infective agents, vaccines, immunosuppressants, anticonvulsants, anti-dementia drugs, prostaglandins, antipyretics, anticycotics, anti-psoriasis agents, antiviral agents, vasodilators or vasoconstrictors, sunscreen preparations (e.g. PABA), antihistamines, hormones such as oestrogen, progesterone or androgens, antiseborrhetic agents, cardiovascular treatment agents such as alpha or beta blockers or Rogaine, vitamins, or any combination thereof.

Particular examples include analgesic agents, such as ibuprofen, indomethacin, diclofenac, acetylsalicylic acid, paracetamol, propranolol, metoprolol, and oxycodone; thyroid release hormone; sex hormones, such as oestragen, progesterone and testosterone; insulin; verapamil; vasopressin; hydrocortisone; scopolamine; nitroglycerine; isosorbide dintirate; anti-histamines, such as terfenadine; clonidine; nicotine; non-steroidal immunosuppressant drugs, such as cyclosporine, methotrexate, azathioprine, mycophenylate, cyclophosphamide, TNF-α antagonists and anti-IL5, -IL4Ra, -IL6, -IL13, -IL17, -IL23 cytokine monoclonal antibodies; anti-convulsants; and drugs for Alzheimer's, dementia and/or Parkinson's disease, such as apamorphine and rivastigmine.

### Treatment, Amelioration or Prevention of Respiratory Diseases or Disorders

The present invention compositions, kits and combinations for the treatment, amelioration or prevention of respiratory diseases or disorders, and a method for the treatment, amelioration or prevention of respiratory diseases or disorders, the method including the administration of a therapeutically effective amount of a composition, a pharmaceutical composition or a combination of the components of a kit as disclosed herein.

### Respiratory Diseases or Disorders

The conditions treatable using the present invention may include lung diseases such as viral infections for example influenza, SARS-CoV or SARS-CoV-2, pulmonary arterial hypertension, pulmonary fibrosis of any cause, bronchiectasis of any cause (including Cystic Fibrosis and Non Cystic Fibrotic Bronchiectasis), interstitial pneumonia of an cause, chronic obstructive pulmonary disease (COPD) (particularly, emphysema, chronic bronchitis), asthma including severe asthma and viral and bacterial induced exacerbations of asthma and refractory (non-reversible) asthma, intra-nasal or pulmonary bacterial infections such as pneumonia, tuberculosis, non-tuberculosis mycobacterial infections, and other bacterial, protozoal and viral lung infections, for example secondary bacterial infections following virus infections of the respiratory tract.

The property of nitric oxide to induce vasodilation characterises some of the treatments using the composition, combination or pharmaceutical composition of the present disclosure and the NOx gas evolved therefrom.

In some embodiments, the respiratory disease or disorder may be associated with the presence of one or more microbes in the subject to be treated. In other words, the respiratory disease or disorder may be associated with one or more microbial infections in the subject. The NOx gas evolved from the composition or pharmaceutical composition may have a biocidal or biostatic effect on a potentially wide range of microorganisms, leading to many anti-microbial treatments. The microbes may, for example, be any one or more selected from bacterial cells, viral particles and/or fungal cells, or microparasites, and may be individual cells, organisms or colonies.

When the microbe is present in a bacterial infection, a fungal infection, viral or microparasitic infection of a human or other animal, the infection may, for example, be in the context of a disease such as the common cold, influenza, tuberculosis, SARS, COVID-19, pneumonia or measles.

The bacterium may be a pathogenic bacterial species. The microbial infection may be an infection caused by a pathogenic bacterial species, including Gram positive and Gram negative, aerobic and anaerobic, antibiotic-sensitive and antibiotic-resistant bacteria.

Examples of bacterial species which may be targeted using the present invention include species of the Actinomyces, Bacillus, Bartonella, Bordetalla, Borrelia, Brucella, Campylobacter, Chlamydia, Chlamydophila, Clostridium, Corynebacterium, Enterococcus, Escherichia, Francisella, Haemophilus, Heliobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, or Yersinia genera. Any combination thereof can also be targeted by the present invention.

The microbe may be a pathogenic species of Corynebacterium, Mycobacterium, Streptococcus, Staphylococcus, Pseudomonas or any combination thereof.

The microbe to be targeted may be selected from Actinomyces israelii, Bacillus anthracis, Bacteroides fragilis, Bordetella pertussis, Borrelia burgdorferi, Borrelia garinii; Borrelia afzelii; Borrelia recurrentis; Brucella abortus; Brucella canis; Brucella melitensis; Brucella suis; Campylobacter jejuni; Chlamydia pneumoniae; Chlamydia trachomatis; Chlamydophila psittaci; Clostridium botulinum; Clostridium difficile; Clostridium perfringens; Clostridium tetani; Corynebacterium diphtheria; Ehrlichia canis; Ehrlichia chaffeensis; Enterococcus faecalis; Enterococcus faecium; Escherichia coli, such as Enterotoxigenic E. coli (ETEC), Enteropathogenic E. coli, Enteroinvasive E. coli (EIEC), and Enterohemorrhagic (EHEC), including E. coli O157:H7; Francisella tularensis; Haemophilus influenza; Helicobacter pylori; Klebsiella pneumoniae; Legionella pneumophila; Leptospira species; Listeria monocytogenes; Mycobacterium leprae; Mycobacterium tuberculosis; Mycobacterium abscessus; Mycobacterium ulcerans; Mycoplasma pneumoniae; Mycobacterium avium; Mycobacterium Kansai; Neisseria gonorrhoeae; Neisseria meningitides; Pseudomonas aeruginosa; Nocardia asteroids; Rickettsia rickettsia; Salmonella typhi; Salmonella typhimurium; Shigella sonnei; Shigella dysenteriae; Staphylococcus aureus; Staphylococcus epidermidis; Staphylococcus saprophyticus; Streptococcus agalactiae; Streptococcus pneumoniae; Streptococcus pyogenes; Streptococcus viridans; Treponema pallidum subspecies pallidum; Vibrio cholera; Yersinia pestis; and any combination thereof.

The microbe may be selected from Chlamydia pneumoniae, Bacillus anthracis, Corynebacterium diphtheria, Haemophilus influenza, Mycobacterium leprae, Mycobacterium tuberculosis, Mycobacterium abscessus, Mycobacterium ulcerans, Pseudomonas aeruginosa, Staphylococcus aureus, Streptococcus pneumoniae, or any combination thereof.

The microbe may be an antibiotic-resistant or antibiotic-sensitive pathogenic bacterial species or an antibiotic-resistant or antibiotic-sensitive strain of a bacterial species. The use of nitric oxide to treat methicillin resistant Staphylococcus aureus (MRSA) and methicillin sensitive Staphylococcus aureus (MSSA) is described, for example, in WO 02/20026, the disclosure of which is incorporated herein by reference. An example of an antibiotic-resistant or antibiotic-sensitive pathogenic bacterial species which may be killed or treated using the present invention is thus methicillin resistant Staphylococcus aureus (MRSA) or methicillin sensitive Staphylococcus aureus (MSSA).

The microbe may be a pathogenic fungal species. The microbial infection may be an infection caused by a pathogenic fungal species, including pathogenic yeasts.

Examples of fungal species which may be targeted using the present invention include species of Aspergillus, Blastomyces, Candida (for example Candida auris), Coccidioides, Paracoccidioides, Cryptosporidium, Cryptococcus (in particular, Cryptococcus neofromans or Cryptococcus gattii), Histoplasma, Mucormycetes, Pneumocystis (for example Pneumocystis jirovecii or carinii), Sporothrix, Talaromyces, or any combination thereof.

Examples of fungal infections include aspergillosis (such as allergic bronchia pulmonary aspergillosis), histoplasmosis, coccidioidomycosis, blastomycosis, paracoccidioidomycosis, mucormycosis, cryptococcosis and infections caused by a pathogenic species of Candida, such as candidiasis.

The microbe may be a virus particle. The infection may be cause by a pathogenic virus.

Examples of viruses which may be targeted using the present invention include influenza viruses, parainfluenza viruses, adenoviruses, noroviruses, rotaviruses, rhinoviruses, coronaviruses, respiratory syncytial virus (RSV), astroviruses, and hepatic viruses. The compositions of the present invention may be used in the treatment or prevention of an infection caused by one of the group selected from H1N1 influenza virus, Infectious Bovine Rhinotracheitis virus, Bovine Respiratory Syncytial virus, Bovine Parainfluenza-3 virus, SARS-CoV, SARS-CoV-2, and any combination thereof.

The invention may be applied to treat a disease or disorder caused by a viral infection.

Respiratory viral infections include influenza, rhinovirus (i.e. common cold virus), respiratory syncytial virus, adenovirus, coronavirus infections, for example, COVID-19, and severe acute respiratory syndrome (SARS), Cytomegalovirus and HSV. Gastrointestinal viral diseases include norovirus infections, rotavirus infections, adenovirus infections and astrovirus infections. Exanthematous viral diseases include measles, rubella, chickenpox, shingles, roseola, smallpox, fifth disease and chikungunya virus disease. Hepatic viral diseases include hepatitis A, hepatitis B, hepatitis C, hepatitis D and hepatitis E.

The microbe may be a parasitic microorganism (microparasite). The infection may be caused by a pathogenic parasitic microorganism. Examples of parasitic microorganisms which may be targeted using the present invention include protozoa.

In particular, the invention may target the protozoa groups of Sarcodina (e.g. amoeba, for example Entamoeba such as Entamoeba histolytica or Entamoeba dispar), Mastigophora (e.g. flagellates, for example Giardia and Leishmania), Ciliophora (e.g. ciliates, for example Balantidium), Sporozoa (e.g. Plasmodium and Cryptosporidium), and any combination thereof.

Parasitic infections that may be treated using the present invention include pulmonary malaria, pulmonary amebiasis, pulmonary babesiosis, pulmonary toxoplasmosis and pulmonary leishmaniasis (e.g. mucocutaneous leishmaniasis).

In particular, the respiratory disease or disorder may be tuberculosis.

### Methods of Administration

The compositions of the present invention are typically administered to a subject shortly after the three constituents, nitrite salt, acid and water, are combined. In this way, the subject may be exposed to the reaction products of the acidification of the nitrite shortly after the reaction is initiated.

The kit as described herein may include the acid and nitrite salt as separate components, particularly if either or both of the acid and nitrite salt are provided as an aqueous solution. Alternatively, the acid and nitrite salt may be solid powders and may be admixed in the kit. In these embodiments, the kit includes instructions with the volume of aqueous media (e.g., water) to be added to the admixed solid powder. In some embodiments, the kit includes a separate container with a fixed volume of aqueous media and instructions to add the fixed volume of aqueous media to generate the buffer system.

Prior to administration, the nitrite salt, acid and aqueous media may then be combined to provide the composition of the present invention. The resulting composition may then be administered to the subject.

In particular embodiments, the composition is administered to a subject by inhalation of an aerosol of the composition (e.g., by nebulisation).

### Subject

The subject may be an animal or human subject. The term “animal” herein generally can include human; however, where the term “animal” appears in the phrase “an animal or human subject” or the like, it will be understood from the context to refer particularly to non-human animals or that the reference to “human” merely particularises the option that the animal may be a human to avoid doubt.

The subject may be a human subject. The human subject may be an infant or adult subject.

The subject may be a vertebrate animal subject. The vertebrate animal may be in the Class Agnatha (jawless fish), Class Chondrichthyes (cartilaginous fish), Class Osteichthyes (bony fish), Class Amphibia (amphibians), Class Reptilia (reptiles), Class Aves (birds), or Class Mammalia (mammals). The subject may be an animal subject in the Class Mammalia or Aves.

The subject may be a domestic species of animal. The domestic species of animal may be one of:
commensals, adapted to a human niche (e.g., dogs, cats, guinea pigs)
prey or farm animals sought or farmed for food (e.g., cows, sheep, pig, goats); and
animals for primarily draft purposes (e.g., horse, camel, donkey)

Examples of domestic animals include, but are not limited to: alpaca, addax, bison, camel, canary, capybara, cat, cattle (including Bali cattle), chicken, collared peccary, deer (including fallow deer, sika deer, thorold's deer, and white-tailed deer), dog, donkey, dove, duck, eland, elk, emu, ferret, gayal, goat, goose, guinea fowl, guinea pig, greater kudu, horse, llama, mink, moose, mouse, mule, muskox, ostrich, parrot, pig, pigeon, quail, rabbit, rat (including the greater cane rat), reindeer, scimitar oryx, sheep, turkey, water buffalo, yak and zebu.

### EXAMPLES

### Example 1: Example Calculation of Buffer Capacity and Relevant Useful Buffer Capacity

Buffer capacity β is calculated using equation (2):

$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) ,$$

as described herein. The relevant useful buffer capacity is calculated as described herein. In this example, we calculate the buffer capacity and the relevant useful buffer capacity for a citric/citrate buffer system (with three pKa values) at different pH values and buffer concentrations. A value of K_{w}=1×10⁻¹⁴ is used.

In the calculation below, the integers K_{w}/[H⁺] and [H⁺] are calculated for each pH value and the integer

$$\frac{{C}_{buf} {K}_{a} [ {H}^{+} ]}{{( {K}_{a} + [ {H}^{+} ] )}^{2}} )$$

is calculated for each pKa value and pH value. The separate three values of

$$\frac{{C}_{buf} {K}_{a} [ {H}^{+} ]}{{( {K}_{a} + [ {H}^{+} ] )}^{2}} )$$

for each pKa are summed, added to the integers K_{w}/[H⁺] and [H⁺] and then multiplied by 2.303 to provide buffer capacity.

The buffer capacity was calculated at pH intervals of 0.01 in order to calculate the Ruβ by taking the integral of buffer capacity β with respect to pH between the pH of the buffer system (pH_i) and a pH of 6.00 using the trapezoidal method at pH intervals of 0.01. The 0.01 capacity intervals are calculated by taking an average of the buffer capacity value at a given pH (e.g., 4.60) and the buffer capacity value at a pH 0.01 higher than the given pH value (e.g., 4.61) and multiplying the average value by the interval value (namely, 0.01). The relevant useful buffer capacity value is then calculated by addition of each integral value between the pH value and 6.00.

Only part of the calculation over the pH range of 4.6 to 6.0 is shown in the tables below, although all intervals between 4.60 and 6.01 were calculated in order provide the relevant useful buffer capacity over the entire range.

FIG. 1 shows a plot of buffer capacity as calculated by equation (2) as described herein for citric/citrate buffer systems across the pH range of 4.0 to 6.0 at buffer concentrations of 0.025 M, 0.05 M, 0.10 M, 0.125 M, 0.15 M and 0.20 M. The buffer capacity increases with increasing buffer concentration. The buffer capacity of the 0.025 M citric/citrate buffer system is not above 0.02 across the pH range. The buffer capacity of the 0.20 M citric/citrate buffer system varies between about 0.10 and 0.14 across the pH range.

FIG. 2 shows a plot of the relevant useful buffer capacity (as the integral of buffer capacity β with respect to pH between the pH of the buffer system and a pH of 6.00 using the trapezoidal method at pH intervals of 0.01) for citric/citrate buffer systems across the pH range of 4.0 to 6.0 at buffer concentrations of 0.025 M, 0.05 M, 0.10 M, 0.125 M, 0.15 M and 0.20 M. The relevant useful buffer capacity generally increases with decreasing pH values. The relevant useful buffer capacity at a given pH generally increases with increasing buffer concentration.

### Calculation of Buffer Capacity and Relevant Buffer Capacity for Citric/Citrate at a Concentration of 0.025 M

| | | | pKa = 3.1 | pKa = 4.7 | pKa = 6.4 | | | |
|---|---|---|---|---|---|---|---|---|
| | | | Ka = 0.0007943 | Ka = 0.0000200 | Ka = 0.0000004 | | 0.01 | |
| | | | [B]Ka[H⁺]/ | [B]Ka[H⁺]/ | [B]Ka[H⁺]/ | Buffer | Capacity | |
| pH | [H⁺] | K_{w}/[H⁺] | (Kₐ + [H⁺])² | (Kₐ + [H⁺])² | (Kₐ + [H⁺])² | Capacity | Integrals | RUB |
| 4.60 | 2.5E−05 | 4.0E−10 | 7.4E−04 | 6.2E−03 | 3.8E−04 | 0.01686 | 0.000169 | 0.020375 |
| 4.61 | 2.5E−05 | 4.1E−10 | 7.3E−04 | 6.2E−03 | 3.9E−04 | 0.01688 | 0.000169 | 0.020207 |
| 4.62 | 2.4E−05 | 4.2E−10 | 7.1E−04 | 6.2E−03 | 4.0E−04 | 0.01689 | 0.000169 | 0.020038 |
| 4.63 | 2.3E−05 | 4.3E−10 | 7.0E−04 | 6.2E−03 | 4.1E−04 | 0.01690 | 0.000169 | 0.019869 |
| 4.64 | 2.3E−05 | 4.4E−10 | 6.8E−04 | 6.2E−03 | 4.2E−04 | 0.01691 | 0.000169 | 0.019700 |
| 4.65 | 2.2E−05 | 4.5E−10 | 6.7E−04 | 6.2E−03 | 4.3E−04 | 0.01692 | 0.000169 | 0.019531 |
| 4.66 | 2.2E−05 | 4.6E−10 | 6.5E−04 | 6.2E−03 | 4.4E−04 | 0.01693 | 0.000169 | 0.019361 |
| 4.67 | 2.1E−05 | 4.7E−10 | 6.4E−04 | 6.2E−03 | 4.5E−04 | 0.01693 | 0.000169 | 0.019192 |
| 4.68 | 2.1E−05 | 4.8E−10 | 6.2E−04 | 6.2E−03 | 4.6E−04 | 0.01693 | 0.000169 | 0.019023 |
| 4.69 | 2.0E−05 | 4.9E−10 | 6.1E−04 | 6.2E−03 | 4.7E−04 | 0.01693 | 0.000169 | 0.018854 |
| 4.70 | 2.0E−05 | 5.0E−10 | 6.0E−04 | 6.3E−03 | 4.8E−04 | 0.01692 | 0.000169 | 0.018684 |
| . . . | . . . | . . . | . . . | . . . | . . . | . . . | . . . | . . . |
| 5.90 | 1.3E−06 | 7.9E−09 | 3.9E−05 | 1.4E−03 | 4.6E−03 | 0.01382 | 0.000138 | 0.001556 |
| 5.91 | 1.2E−06 | 8.1E−09 | 3.9E−05 | 1.4E−03 | 4.6E−03 | 0.01388 | 0.000139 | 0.001418 |
| 5.92 | 1.2E−06 | 8.3E−09 | 3.8E−05 | 1.3E−03 | 4.7E−03 | 0.01394 | 0.000140 | 0.001279 |
| 5.93 | 1.2E−06 | 8.5E−09 | 3.7E−05 | 1.3E−03 | 4.7E−03 | 0.01399 | 0.000140 | 0.001139 |
| 5.94 | 1.1E−06 | 8.7E−09 | 3.6E−05 | 1.3E−03 | 4.8E−03 | 0.01405 | 0.000141 | 0.000999 |
| 5.95 | 1.1E−06 | 8.9E−09 | 3.5E−05 | 1.3E−03 | 4.8E−03 | 0.01412 | 0.000141 | 0.000858 |
| 5.96 | 1.1E−06 | 9.1E−09 | 3.4E−05 | 1.2E−03 | 4.9E−03 | 0.01418 | 0.000142 | 0.000716 |
| 5.97 | 1.1E−06 | 9.3E−09 | 3.4E−05 | 1.2E−03 | 4.9E−03 | 0.01424 | 0.000143 | 0.000574 |
| 5.98 | 1.0E−06 | 9.5E−09 | 3.3E−05 | 1.2E−03 | 5.0E−03 | 0.01430 | 0.000143 | 0.000432 |
| 5.99 | 1.0E−06 | 9.8E−09 | 3.2E−05 | 1.2E−03 | 5.0E−03 | 0.01436 | 0.000144 | 0.000288 |
| 6.00 | 1.0E−06 | 1.0E−08 | 3.1E−05 | 1.1E−03 | 5.1E−03 | 0.01442 | 0.000144 | 0.000144 |
| 6.01 | 9.8E−07 | 1.0E−08 | 3.1E−05 | 1.1E−03 | 5.1E−03 | 0.01448 | | |

### Calculation of Buffer Capacity and Relevant Buffer Capacity for Citric/Citrate at a Concentration of 0.20 M

| | | | pKa = 3.1 | pKa = 4.7 | pKa = 6.4 | | | |
|---|---|---|---|---|---|---|---|---|
| | | | Ka = 0.0007943 | Ka = 0.0000200 | Ka = 0.0000004 | | 0.01 | |
| | | | [B]Ka[H⁺]/ | [B]Ka[H⁺]/ | [B]Ka[H⁺]/ | Buffer | Capacity | |
| pH | [H⁺] | K_{w}/[H⁺] | (Kₐ + [H⁺])² | (Kₐ + [H⁺])² | (Kₐ + [H⁺])² | Capacity | Integrals | RUB |
| 4.60 | 2.5E−05 | 4.0E−10 | 5.9E−03 | 4.9E−02 | 3.1E−03 | 0.13446 | 0.001345 | 0.162834 |
| 4.61 | 2.5E−05 | 4.1E−10 | 5.8E−03 | 4.9E−02 | 3.1E−03 | 0.13461 | 0.001347 | 0.161489 |
| 4.62 | 2.4E−05 | 4.2E−10 | 5.7E−03 | 5.0E−02 | 3.2E−03 | 0.13474 | 0.001348 | 0.160142 |
| 4.63 | 2.3E−05 | 4.3E−10 | 5.6E−03 | 5.0E−02 | 3.3E−03 | 0.13485 | 0.001349 | 0.158795 |
| 4.64 | 2.3E−05 | 4.4E−10 | 5.4E−03 | 5.0E−02 | 3.4E−03 | 0.13494 | 0.001350 | 0.157446 |
| 4.65 | 2.2E−05 | 4.5E−10 | 5.3E−03 | 5.0E−02 | 3.4E−03 | 0.13501 | 0.001350 | 0.156096 |
| 4.66 | 2.2E−05 | 4.6E−10 | 5.2E−03 | 5.0E−02 | 3.5E−03 | 0.13506 | 0.001351 | 0.154746 |
| 4.67 | 2.1E−05 | 4.7E−10 | 5.1E−03 | 5.0E−02 | 3.6E−03 | 0.13508 | 0.001351 | 0.153395 |
| 4.68 | 2.1E−05 | 4.8E−10 | 5.0E−03 | 5.0E−02 | 3.7E−03 | 0.13509 | 0.001351 | 0.152044 |
| 4.69 | 2.0E−05 | 4.9E−10 | 4.9E−03 | 5.0E−02 | 3.8E−03 | 0.13508 | 0.001351 | 0.150693 |
| 4.70 | 2.0E−05 | 5.0E−10 | 4.8E−03 | 5.0E−02 | 3.8E−03 | 0.13504 | 0.001350 | 0.149343 |
| . . . | . . . | . . . | . . . | . . . | . . . | . . . | . . . | . . . |
| 5.90 | 1.3E−06 | 7.9E−09 | 3.2E−04 | 1.1E−02 | 3.7E−02 | 0.11052 | 0.001108 | 0.012447 |
| 5.91 | 1.2E−06 | 8.1E−09 | 3.1E−04 | 1.1E−02 | 3.7E−02 | 0.11099 | 0.001112 | 0.011339 |
| 5.92 | 1.2E−06 | 8.3E−09 | 3.0E−04 | 1.1E−02 | 3.7E−02 | 0.11146 | 0.001117 | 0.010227 |
| 5.93 | 1.2E−06 | 8.5E−09 | 2.9E−04 | 1.1E−02 | 3.8E−02 | 0.11194 | 0.001122 | 0.009110 |
| 5.94 | 1.1E−06 | 8.7E−09 | 2.9E−04 | 1.0E−02 | 3.8E−02 | 0.11242 | 0.001127 | 0.007988 |
| 5.95 | 1.1E−06 | 8.9E−09 | 2.8E−04 | 1.0E−02 | 3.9E−02 | 0.11290 | 0.001131 | 0.006861 |
| 5.96 | 1.1E−06 | 9.1E−09 | 2.8E−04 | 9.9E−03 | 3.9E−02 | 0.11339 | 0.001136 | 0.005730 |
| 5.97 | 1.1E−06 | 9.3E−09 | 2.7E−04 | 9.7E−03 | 4.0E−02 | 0.11387 | 0.001141 | 0.004594 |
| 5.98 | 1.0E−06 | 9.5E−09 | 2.6E−04 | 9.5E−03 | 4.0E−02 | 0.11436 | 0.001146 | 0.003452 |
| 5.99 | 1.0E−06 | 9.8E−09 | 2.6E−04 | 9.3E−03 | 4.0E−02 | 0.11484 | 0.001151 | 0.002306 |
| 6.00 | 1.0E−06 | 1.0E−08 | 2.5E−04 | 9.1E−03 | 4.1E−02 | 0.11532 | 0.001156 | 0.001156 |
| 6.01 | 9.8E−07 | 1.0E−08 | 2.5E−04 | 8.9E−03 | 4.1E−02 | 0.11580 | | |

### Example 2: Antimicrobial Activity of NO-Releasing Formulations

A number of NO-releasing formulations were tested to determine the antimicrobial activity of these formulations against Psuedomonas aeruinosa NCTC 13618 using a 96-well suspension method, as described below.

### Methodology

1. One hundred microlitres of twice the desired final concentration of the sample was aliquoted into the relevant wells of a 96-well plate. Two concentrations of each formulation were tested in triplicate in each plate.
2. Bacterial suspensions were prepared to 1×10⁸±5×10⁷ CFUmL⁻¹ in cation-adjusted Mueller Hinton II broth (CAMHIIB).
3. The prepared 96-well agent plates were inoculated with 100 μL/well of the bacterial suspensions to a final concentration of 5×10⁵±3×10⁵ CFUmL⁻¹ and the inoculum was enumerated.
4. Sterility controls, negative controls and positive controls were also included.
5. Following inoculation, the plates were added to a temperature-controlled plate reader for 24 hours. The plate reader took optical density (OD) readings at 0, 4, 8, 12, 20 and 24 hours.
6. Following the 24-hour period, the plates were removed from the plate reader and the suspensions were mixed, serially diluted and quantified to provide the reported reduction results below.

### Formulations

| | Nitrite | Mannitol | Citric/Citrate | |
|---|---|---|---|---|
| Formu- | concentration | concentration | concentration | |
| lation | (M) | (M) | (M) | pH |
| 1 | — | — | 0.025 | 5.0 |
| 2 | — | — | 0.05 | 5.0 |
| 3 | — | — | 0.10 | 5.0 |
| 4 | — | — | 0.125 | 5.0 |
| 5 | — | — | 0.15 | 5.0 |
| 6 | — | — | 0.20 | 5.0 |
| 7 | — | — | 0.15 | 4.6 |
| 8 | — | — | 0.15 | 4.8 |
| 9 | — | — | 0.15 | 5.2 |
| 10 | — | — | 0.15 | 5.4 |
| 11 | 0.15 | 0.05 | 0.025 | 4.8 |
| 12 | 0.15 | 0.05 | 0.05 | 4.8 |
| 13 | 0.15 | 0.05 | 0.10 | 4.8 |
| 14 | 0.15 | 0.05 | 0.125 | 4.8 |
| 15 | 0.15 | 0.05 | 0.15 | 4.8 |
| 16 | 0.15 | 0.05 | 0.20 | 4.8 |
| 17 | 0.15 | 0.05 | 0.025 | 5.0 |
| 18 | 0.15 | 0.05 | 0.05 | 5.0 |
| 19 | 0.15 | 0.05 | 0.10 | 5.0 |
| 20 | 0.15 | 0.05 | 0.125 | 5.0 |
| 21 | 0.15 | 0.05 | 0.15 | 5.0 |
| 22 | 0.15 | 0.05 | 0.20 | 5.0 |
| 23 | 0.15 | 0.05 | 0.025 | 5.4 |
| 24 | 0.15 | 0.05 | 0.05 | 5.4 |
| 25 | 0.15 | 0.05 | 0.10 | 5.4 |
| 26 | 0.15 | 0.05 | 0.125 | 5.4 |
| 27 | 0.15 | 0.05 | 0.15 | 5.4 |
| 28 | 0.15 | 0.05 | 0.20 | 5.4 |
| 29 | 0.22 | 0.05 | 0.025 | 4.8 |
| 30 | 0.22 | 0.05 | 0.05 | 4.8 |
| 31 | 0.22 | 0.05 | 0.10 | 4.8 |
| 32 | 0.22 | 0.05 | 0.125 | 4.8 |
| 33 | 0.22 | 0.05 | 0.15 | 4.8 |
| 34 | 0.22 | 0.05 | 0.20 | 4.8 |
| 35 | 0.22 | 0.05 | 0.025 | 5.0 |
| 36 | 0.22 | 0.05 | 0.05 | 5.0 |
| 37 | 0.22 | 0.05 | 0.10 | 5.0 |
| 38 | 0.22 | 0.05 | 0.125 | 5.0 |
| 39 | 0.22 | 0.05 | 0.15 | 5.0 |
| 40 | 0.22 | 0.05 | 0.20 | 5.0 |
| 41 | 0.22 | 0.05 | 0.025 | 5.4 |
| 42 | 0.22 | 0.05 | 0.05 | 5.4 |
| 43 | 0.22 | 0.05 | 0.10 | 5.4 |
| 44 | 0.22 | 0.05 | 0.125 | 5.4 |
| 45 | 0.22 | 0.05 | 0.15 | 5.4 |
| 46 | 0.22 | 0.05 | 0.20 | 5.4 |

### Results

| | 12.50% | 6.25% | 12.50% | 6.25% |
|---|---|---|---|---|
| | Dilution | Dilution | Dilution | Dilution |
| Formu- | Reduction | Reduction | Reduction | Reduction |
| lation | (Log10 CFU/mL) | (Log10 CFU/mL) | SD | SD |
| 1 | 0.01 | 0.14 | 0.13 | 0.09 |
| 2 | 0.05 | 0.15 | 0.63 | 0.55 |
| 3 | 0.44 | 0.03 | 0.39 | 0.03 |
| 4 | 0.03 | 0.46 | 0.03 | 0.46 |
| 5 | 0.38 | 0.04 | 0.41 | 0.19 |
| 6 | 0.43 | 0.16 | 0.15 | 0.05 |
| 7 | 6.71 | 2.44 | 0.00 | 0.09 |
| 8 | 0.38 | 0.28 | 0.04 | 0.13 |
| 9 | 0.23 | 0.21 | 0.45 | 0.52 |
| 10 | 0.07 | 0.19 | 0.15 | 0.28 |
| 11 | 8.90 | 4.72 | 0.00 | 0.38 |
| 12 | 8.90 | 8.90 | 0.00 | 0.00 |
| 13 | 7.81 | 8.24 | 1.33 | 1.40 |
| 14 | 8.61 | 8.61 | 0.00 | 0.00 |
| 15 | 9.04 | 9.04 | 0.00 | 0.00 |
| 16 | 8.61 | 8.13 | 0.00 | 0.46 |
| 17 | 6.20 | 0.18 | 2.46 | 0.25 |
| 18 | 9.04 | 1.64 | 0.00 | 0.36 |
| 19 | 9.04 | 6.23 | 0.00 | 1.20 |
| 20 | 9.04 | 5.94 | 0.00 | 0.46 |
| 21 | 8.61 | 8.61 | 0.00 | 0.00 |
| 22 | 8.61 | 8.61 | 0.00 | 0.00 |
| 23 | 0.09 | 0.16 | 0.25 | 0.10 |
| 24 | 4.97 | 0.16 | 0.08 | 0.37 |
| 25 | 4.52 | 0.20 | 0.35 | 0.04 |
| 26 | 5.17 | 0.54 | 0.22 | 0.15 |
| 27 | 5.02 | 0.60 | 0.92 | 0.23 |
| 28 | 8.74 | 2.13 | 0.63 | 0.94 |
| 29 | 3.96 | 0.37 | 0.87 | 0.07 |
| 30 | 9.10 | 3.91 | 0.00 | 0.05 |
| 31 | 9.10 | 7.89 | 0.00 | 1.19 |
| 32 | 9.10 | 7.80 | 0.00 | 1.19 |
| 33 | 9.10 | 9.10 | 0.00 | 0.00 |
| 34 | 9.10 | 7.72 | 0.00 | 0.35 |
| 35 | 3.36 | 0.43 | 0.04 | 0.03 |
| 36 | 9.10 | 0.51 | 0.00 | 0.05 |
| 37 | 8.61 | 8.61 | 0.00 | 0.00 |
| 38 | 8.61 | 6.60 | 0.00 | 0.92 |
| 39 | 8.61 | 6.69 | 0.00 | 0.77 |
| 40 | 8.61 | 8.06 | 0.00 | 0.09 |
| 41 | 3.25 | 0.14 | 0.20 | 0.16 |
| 42 | 6.78 | 0.18 | 2.03 | 0.25 |
| 43 | 8.61 | 2.53 | 0.00 | 2.01 |
| 44 | 8.61 | 3.60 | 0.00 | 0.44 |
| 45 | 8.61 | 3.31 | 0.00 | 0.04 |
| 46 | 8.61 | 3.54 | 0.00 | 0.04 |

FIGS. 3 and 4 show the microbial reduction (Log 10 CFU/mL) at a 6.25% dilution for formulations including 0.15 M nitrite and 0.22 M nitrite, respectively.

### Example 3: Antimicrobial Activity of NO-Releasing Formulations

A number of NO-releasing formulations were tested to determine the antimicrobial activity of these formulations against Mycobacterium abscessus ATCC 19977 using a 96-well suspension method, as described below.

### Methodology

1. Discrete colonies Mycobacterium abscessus ATCC 19977 were harvested and re-suspended in cation-adjusted Mueller Hinton II broth (CAMHIBB). The suspension was adjusted to produce a final concentration of 5×10⁷±3×10⁷ CFUmL⁻¹. The inoculum concentration was enumerated by performing 10-fold dilutions in Phosphate Buffered Saline (PBS) and plating out the resulting suspensions onto Middlebrook agar with 10% OADC Enrichment solution (MBA).
2. Test agents were prepared according to the formulations below. Neat test agents were then diluted to achieve test agent concentrations of 25.0%.
3. One hundred microliters of each prepared test agent concentration was aliquoted into the relevant wells of a 96-well plate and mixed with 100 μL of the bacterial suspension to achieve final test concentrations of 12.5% in triplicate.
4. Sterility controls, negative controls and positive controls were also included.
5. Following inoculations, the plates were added to a temperature-controlled-plate reader for 72 hours. The plate reader took optical density (OD) readings at 0, 4, 8, 12, 24, 48 and 72 hours.
6. Following the 72-hour period, the plates were removed from the plate reader and the suspension were serially diluted and quantified to provide the reported reduction results below.
7. Testing was repeated three more times for a total of four biological replicates for each formulation.

### Formulations

| | Nitrite | Mannitol | Citric/Citrate | |
|---|---|---|---|---|
| Formu- | concentration | concentration | concentration | |
| lation | (M) | (M) | (M) | pH |
| 1 | 0.15 | 0.05 | 0.1 | 5.4 |
| 2 | 0.15 | 0.005 | 0.1 | 4.6 |
| 3 | 0.15 | 0.005 | 0.1 | 4.8 |
| 4 | 0.15 | 0.005 | 0.1 | 5.0 |
| 5 | 0.15 | 0.005 | 0.1 | 5.4 |
| 6 | 0.15 | 0.005 | 0.15 | 4.6 |
| 7 | 0.15 | 0.005 | 0.15 | 4.8 |
| 8 | 0.15 | 0.005 | 0.15 | 5.0 |
| 9 | 0.15 | 0.005 | 0.15 | 5.4 |
| 10 | 0.22 | 0.005 | 0.15 | 4.6 |
| 11 | 0.22 | 0.005 | 0.15 | 4.8 |
| 12 | 0.22 | 0.005 | 0.15 | 5.0 |
| 13 | 0.22 | 0.005 | 0.15 | 5.4 |
| 14 | 0.22 | 0.005 | 0.1 | 4.6 |
| 15 | 0.22 | 0.005 | 0.1 | 4.8 |
| 16 | 0.22 | 0.005 | 0.1 | 5.0 |

### Results

| | 12.50% Dilution | |
|---|---|---|
| | Reduction | 12.50% Dilution |
| Formulation | (Log10 CFU/mL) | Reduction SD |
| 1 | 0.00 | 0.00 |
| 2 | 6.41 | 1.22 |
| 3 | 2.88 | 1.24 |
| 4 | 1.13 | 0.53 |
| 5 | 0.21 | 0.45 |
| 6 | 7.75 | 0.79 |
| 7 | 7.20 | 1.21 |
| 8 | 5.16 | 2.78 |
| 9 | 0.20 | 0.87 |
| 10 | 7.96 | 0.00 |
| 11 | 7.96 | 0.00 |
| 12 | 6.52 | 1.70 |
| 13 | 0.81 | 1.04 |
| 14 | 7.16 | 1.25 |
| 15 | 4.44 | 3.28 |
| 16 | 2.05 | 1.35 |

1. A nitric oxide-generating composition for use in the treatment, amelioration or prevention of respiratory diseases or disorders, the composition comprising:
a) one or more nitrite salts; and
b) a buffer system comprising at least one acid and at least one conjugate base and water, wherein the buffer system has a pH in the range of 4.6 to 6.0, and wherein the buffer capacity β of the buffer system is at least 0.06 as calculated by the equation (2):
$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$
where:
K_{w} is the water dissociation equilibrium constant at 25° C.;
[H⁺] is the concentration of hydrogen ions, based on the pH of buffer system;
C_{buf} is the buffer concentration in the composition; and
Kₐ is the dissociation constant of the acid at 25° C.

2. The nitric oxide generating composition for use according to claim 1 wherein the buffer capacity β of the buffer system is at least 0.075 as calculated by the equation (2).

3. A nitric oxide-generating composition for use in the treatment, amelioration or prevention of respiratory diseases or disorders, the composition comprising:
a) One or more nitrite salts; and
b) A buffer system comprising at least one acid, at least one conjugate base and water, wherein the buffer system has a pH in the range of 4.6 to 6.0, and wherein the relevant useful buffer capacity RUβ of the buffer system is at least 0.04, where the Ruβ is defined as in equation (3):
$$RU β = {\int }_{pH \_ i}^{pH = 6.} β ( pH ) d ( pH ) \qquad ( 3 )$$
as the integral of β with respect to pH between the pH of the buffer system and a pH of 6.00 using the trapezoidal method at pH intervals of 0.01 and β is the buffer capacity of the buffer system as calculated by the equation (2):
$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$
where:
K_{w} is the water dissociation equilibrium constant at 25° C.;
[H⁺] is the concentration of hydrogen ions, based on the pH of the buffer system;
C_{buf} is the buffer concentration in the composition; and
Kₐ is the dissociation constant of the acid at 25° C.

4. The nitric oxide-generating composition for use according to claim 3, wherein the relevant useful buffer capacity Ruβ of the buffer system is at least 0.05.

5. The nitric oxide-generating composition for use according to claim 3, wherein the buffer system includes at least one acid is selected from one or more carboxylic acids and one or more organic non-carboxylic reducing acids.

6. The nitric oxide-generating composition according to claim 5, wherein the one or more organic carboxylic acids are selected salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, β-hydroxypropanoic acid, β-hydroxybutyric acid, β-hydroxy-β-butyric acid, naphthoic acid, oleic acid, palmitic acid, pamoic (emboic) acid, stearic acid, malonic acid, succinic acid, fumaric acid, glucoheptonic acid, glucuronic acid, lactobioic acid, cinnamic acid, pyruvic acid, orotic acid, glyceric acid, glycyrrhizic acid, sorbic acid, hyaluronic acid, alginic acid, oxalic acid, salts thereof, and combinations thereof.

7. The nitric oxide-generating composition according to claim 5, wherein the one or more organic non-carboxylic reducing acids are selected from selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid, 6-O-dodecanedioyl ascorbic acid, L-Ascorbic acid 2-phosphate and 2-O-alpha-D-Glucopyranosyl-L-ascorbic acid; acidic reductones such as reductic acid; erythorbic acid; salts thereof; and combinations thereof.

8. The nitric oxide-generating composition for use according to claim 3, wherein the buffer system includes citric acid and citrate.

9. The nitric oxide-generating composition for use according to claim 8 wherein the buffer concentration is at least 0.075 M and optionally no more than 0.5 M.

10. The nitric oxide-generating composition for use according to claim 3, wherein the composition further includes an organic polyol selected from the group consisting of erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof.

11. The nitric oxide-generating composition for use according to claim 3, wherein the one or more nitrite salts is selected from composition LiNO₂, NaNO₂, KNO₂, RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof.

12. The nitric oxide-generating composition for use according to claim 3, wherein the molarity of the nitrite salt in the composition is in the range of 0.001 M to 2.0 M.

13. A kit for providing a nitric oxide-generating composition for use in the treatment, amelioration or prevention of respiratory diseases or disorders, the kit including:
a) a nitrite salt component including one or more nitrite salts;
b) an acid component including at least one acid; and
wherein the kit further includes at least one conjugate base to form a buffer system with the acid component such that the buffer system has a pH of 4.6 to 6.0, and wherein the buffer capacity β of the buffer system is at least 0.06 as calculated by the equation (2):
$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$
where:
K_{w} is the water dissociation equilibrium constant at 25° C.;
[H⁺] is the concentration of hydrogen ions, based on the pH of the buffer system;
C_{buf} is the buffer concentration in the nitric oxide-generating composition; and
Kₐ is the dissociation constant of the acid at 25° C.

14. A kit for providing a nitric oxide-generating composition for use in the treatment, amelioration or prevention of respiratory diseases or disorders the kit including:
a) a nitrite salt component including one or more nitrite salts; and
b) an acid component including at least one acid; and
wherein the kit further includes at least one conjugate base to form a buffer system with the acid component such that the buffer system has a pH of 4.6 to 6.0, and the relevant useful buffer capacity RUβ of the buffer system is at least 0.04, where the Ruβ is defined as in equation (3)
$$R U β = {\int }_{pH \_ i}^{pH = 6.} β ( pH ) d ( pH ) \qquad ( 3 )$$
as the integral of β with respect to pH between the pH of the buffer system and a pH of 6.00 using the trapezoidal method at pH intervals of 0.01 and β is the buffer capacity of the buffer system as calculated by the equation (2):
$$β = 2.303 ( \frac{{K}_{w}}{[ {H}^{+} ]} + [ {H}^{+} ] + \sum \frac{{C}_{buf} {K}_{a} [ {H}^{+} ]}{{( {K}_{a} + [ {H}^{+} ] )}^{2}} ) , \qquad ( 2 )$$
where:
K_{w} is the water dissociation equilibrium constant at 25° C.;
[H⁺] is the concentration of hydrogen ions, based on the pH of the buffer system;
C_{buf} is the buffer concentration in the nitric oxide-generating composition; and
Kₐ is the dissociation constant of the acid at 25° C.

15. The kit for use according to claim 13, wherein the nitrite salt source component is an aqueous solution including one or more nitrite salts and/or the acid component is an aqueous solution including an acid.

16. A nitric oxide-generating composition for use in the treatment, amelioration or prevention of respiratory diseases or disorders, the composition being formed from a combination of the nitrite salt component and the acid component of the kit of claim 13.

17. A method of the treatment, amelioration or prevention of respiratory diseases or disorders, comprising:
administering to a subject in need thereof a composition of claim 1.

18. (canceled)

19. The method of claim 17, wherein the respiratory disease or disorder is associated with one or more microbial infections in a subject.

20. The nitric oxide-generating composition for use according to claim 1, wherein the buffer system includes at least one acid is selected from one or more carboxylic acids and one or more organic non-carboxylic reducing acids.

21. The nitric oxide-generating composition according to claim 20, wherein the one or more organic carboxylic acids are selected salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, β-hydroxypropanoic acid, β-hydroxybutyric acid, β-hydroxy-β-butyric acid, naphthoic acid, oleic acid, palmitic acid, pamoic (emboic) acid, stearic acid, malonic acid, succinic acid, fumaric acid, glucoheptonic acid, glucuronic acid, lactobioic acid, cinnamic acid, pyruvic acid, orotic acid, glyceric acid, glycyrrhizic acid, sorbic acid, hyaluronic acid, alginic acid, oxalic acid, salts thereof, and combinations thereof.

22. The nitric oxide-generating composition according to claim 20, wherein the one or more organic non-carboxylic reducing acids are selected from selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid, 6-O-dodecanedioyl ascorbic acid, L-Ascorbic acid 2-phosphate and 2-O-alpha-D-Glucopyranosyl-L-ascorbic acid; acidic reductones such as reductic acid; erythorbic acid; salts thereof; and combinations thereof.

23. The nitric oxide-generating composition for use according to claim 1, wherein the buffer system includes citric acid and citrate.

24. The nitric oxide-generating composition for use according to claim 23, wherein the buffer concentration is at least 0.075 M and optionally no more than 0.5 M.

25. The nitric oxide-generating composition for use according to claim 1, wherein the composition further includes an organic polyol selected from the group consisting of erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof.

26. The nitric oxide-generating composition for use according to claim 1, wherein the one or more nitrite salts is selected from composition LiNO₂, NaNO₂, KNO₂, RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof.

27. The nitric oxide-generating composition for use according to claim 1, wherein the molarity of the nitrite salt in the composition is in the range of 0.001 M to 2.0 M.

28. The kit for use according to claim 14, wherein the nitrite salt source component is an aqueous solution including one or more nitrite salts and/or the acid component is an aqueous solution including an acid.

29. A nitric oxide-generating composition for use in the treatment, amelioration or prevention of respiratory diseases or disorders, the composition being formed from a combination of the nitrite salt component and the acid component of the kit of claim 16.

30. A method of the treatment, amelioration or prevention of respiratory diseases or disorders, comprising:
administering to a subject in need thereof a composition of claim 3.

31. The method of claim 30, wherein the respiratory disease or disorder is associated with one or more microbial infections in a subject.
